(19) United States
(12) Patent Application Publication
(10) Pub. No.: US 2026/0048064 A1
(43) Pub. Date: Feb. 19, 2026

(54) AMORPHOUS SOLID DISPERSIONS COMPRISING NAPORAFENIB

(71) Applicant: Novartis AG, Basel (CH)

(72) Inventors: Oliver GRANER, Basel (CH); Cornelius Stephan HARLACHER, Basel (CH); Jennifer Claire HOOTON, Basel (CH); Raman IYER, East Hanover, NJ (US); Vijay SETHURAMAN, Fremont, CA (US)

(21) Appl. No.: 19/101,494

(22) PCT Filed: Aug. 9, 2023
(86) PCT No.: PCT/IB2023/000477

§ 371 Date: Feb. 5, 2025

Related U.S. Application Data
(60) Provisional application No. 63/370,989, filed on Aug. 10, 2022.

(51) Int. Cl. A61K 31/5377; A61K 9/20

(52) U.S. Cl. CPC A61K 31/5377; A61K 9/2009; A61K 9/2013; A61K 9/2018; A61K 9/2054

(57) ABSTRACT

The present invention relates to the field of pharmacy, particularly to a pharmaceutical composition comprising N (3-(2-(2-hydroxyethoxy)-6-morpholinopyridin-4-yl)-4-methylphenyl)-2- (trifluoromethyl)isonicotinanmide, or a pharmaceutically acceptable salt thereof. The present invention also provides a process for preparing said pharmaceutical compositions for oral administration and methods of treatment with said pharmaceutical compositions.

### CROSS-REFERENCE

This application claims the benefit of U.S. Provisional Application Ser. No. 63/370,989 filed Aug. 10, 2022; which is hereby incorporated by reference in its entirety.

### FIELD OF THE INVENTION

The present invention provides solid amorphous dispersions comprising N-(3-(2-(2-hydroxyethoxy)-6-morpholinopyridin-4-yl)-4-methylphenyl)-2-(trifluoromethyl)isonicotinamide (Compound A), or a pharmaceutically acceptable salt thereof, and one or more stabilizing polymers. The present invention also provides pharmaceutical compositions or dosage forms comprising the amorphous solid dispersions, processes for preparing the same and methods of treatment using the same. The present invention also provides these pharmaceutical compositions for oral administration.

### BACKGROUND OF THE INVENTION

The RAS/RAF/MEK/ERK or MAPK pathway is a key signalling cascade that drives cell proliferation, differentiation, and survival. Dysregulation of this pathway underlies many instances of tumorigenesis. Aberrant signalling or inappropriate activation of the MAPK pathway has been shown in multiple tumor types, including melanoma, lung, and pancreatic cancer, and can occur through several distinct mechanisms, including activating mutations in RAS and BRAF. RAS is a superfamily of GTPases, and includes KRAS (v-Ki-ras2 Kirsten rat sarcoma viral oncogene homolog), which is a regulated signalling protein that can be turned on (activated) by various single-point mutations, which are known as gain of function mutations. The MAPK pathway is frequently mutated in human cancer with KRAS and BRAF mutations being among the most frequent (approximately 30%).

N-(3-(2-(2-hydroxyethoxy)-6-morpholinopyridin-4-yl)-4-methylphenyl)-2-(trifluoromethyl)isonicotinamide (Compound A) was originally described in WO 2014/151616 as the compound of Example 1156. It is a Raf inhibitor, particularly a CRAF- and BRAF-inhibitor, having the structure of Formula I.

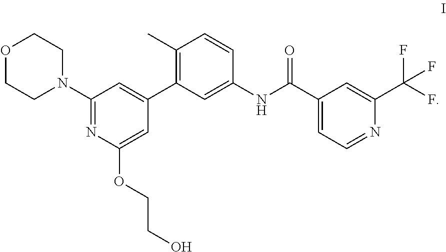 [SMILES: Cc1ccc(NC(=O)c2ccnc(C(F)(F)F)c2)cc1-c1cc(OCCO)nc(N2CCOCC2)c1]

Various crystalline forms, including the monohydrate H_{A} form, of the compound of Formula I, or Compound A, are described in WO/2020/230028. Compound A is also known as “naporafenib.” Compound A may be useful in the treatment of various cancers, in particular in the treatment of cancers harboring MAPK pathway alterations such as KRAS-mutant NSCLC (non-small cell lung cancer), KRAS-mutant pancreatic cancer (e.g., KRAS-mutant pancreatic ductal adenocarcinoma (PDAC)), KRAS-mutant CRC (colorectal cancer), and NRAS-mutant melanoma.

There is a need to formulate Compound A into pharmaceutical compositions, especially oral pharmaceutical dosage forms, such that the therapeutic benefits of the compound may be delivered to a patient in need thereof. The physiochemical properties of the therapeutic compound pose a challenge to resolving this need. Compound A is poorly soluble in aqueous media and has a high permeability lending towards potential solubility and bioavailability issues that need to be addressed in the development of a pharmaceutical dosage form comprising naporafenib. Thus, an object of the present invention is to provide exemplary solutions for making pharmaceutical compositions comprising naporafenib in the form of a solid oral dosage form that may be ingested by a patient.

### BRIEF DESCRIPTION OF THE FIGURES

FIG. 1A depicts drug substance particle morphology for Compound A as the free base—anhydrate (NXA).

FIG. 1B depicts drug substance particle morphology for Compound A as the free base—monohydrate (NXB).

FIG. 2 illustrates a representative process flow diagram for manufacturing 600 mg/g granules of Compound A (API) and the addition of extra-granular components for manufacturing film coated tablets of Compound A (API).

### SUMMARY OF THE INVENTION

As every active pharmaceutical ingredient (API) has its own physical, chemical, and pharmacological characteristics, a suitable pharmaceutical composition and dosage form has to be individually designed for every new API.

The design of a pharmaceutical composition, a pharmaceutical dosage form, as well as a commercially viable process to prepare the pharmaceutical composition, for a Raf inhibitor such as N-(3-(2-(2-hydroxyethoxy)-6-morpholinopyridin-4-yl)-4-methylphenyl)-2-(trifluoromethyl)isonicotinamide (Compound A), as a pharmaceutically acceptable salt thereof, or as the free base, is challenging. This Raf inhibitor is difficult to formulate due to its physicochemical properties, e.g., low solubility, high permeability, and susceptibility to degradation at certain pH conditions and temperatures. These properties affect the pharmacokinetics, the bioavailability and the manufacturing process of formulations comprising said Raf inhibitor of the present invention.

Accordingly, a suitable and robust solid pharmaceutical composition overcoming the above problems needs to be developed. The invention provides a pharmaceutical composition with enhanced drug dissolution and increased absorption. The pharmaceutical composition may also provide an increase of bioavailability and/or a decrease of patient to patient variability. Furthermore, the invention provides a process for making the pharmaceutical composition, wherein such process provides an ease of scale-up, a robust processing method and economic advantages.

The present invention aims to provide a formulation of Compound A that minimizes the size and/or number of tablets or capsules required for the therapeutically effective dose, ideally to fewer than 4 tablets or capsules, preferably only one or two tablet(s) or capsule(s).

In terms of the aim of increasing the therapeutic potential of Compound A, the inventors sought to increase the therapeutic potential by achieving an increase in the bioavailability of Compound A in a formulation that permitted sufficiently high drug loading (e.g., greater than 5%). In distinct embodiments, the drug loading will be at least 5%, 10%, 15%, 20%, 25%, 30%, 35%, 40%, 45%, 50%, 55%, 60%, 65%, 70%, 75% or 80%. It will be appreciated that the greater the drug loading the greater the likelihood of instability. It is therefore not a trivial task to achieve increased drug loading whilst maintaining physical and chemical stability of the resulting drug product.

The inventors discovered that solid dispersion formulations, for example, polymer stabilized amorphous solid dispersion (PSASD) formulations, address one or more of the aims stated above.

The inventors surprisingly found that the therapeutic potential of Compound A can be increased by formulating Compound A as an amorphous solid dispersion with one or more stabilizing polymers. The amorphous solid dispersion of the present invention allows for greater solubility, faster rate of dissolution, and improved bioavailability of Compound A. Amorphous solid dispersion formulations of Compound A with the stabilizing polymer hypromellose were found to be particularly suitable in yielding physically and chemically stable pharmaceutical compositions at high drug loads (e.g., up to 80%) of Compound A. The inventors also found that amorphous solid dispersion formulations prepared with Compound A in the monohydrate form, as compared with Compound A in the anhydrous form, allowed for doubling the drug load in the solid dispersion (e.g., from about 30 to 60%) and a reduction in the tablet size (e.g., about 70% smaller).

In view of the above-mentioned difficulties and considerations, it was not trivial to arrive at a stable pharmaceutical composition which improves the solubility and bioavailability of Compound A, and which is also suitable for manufacturing at a commercial scale.

Aspects, advantageous features and preferred embodiments of the present invention summarized in the following items, respectively alone or in combination, contribute to solving the aims of the invention.

Item A1. An amorphous solid dispersion comprising Compound A, or a pharmaceutically acceptable salt thereof, and one or more stabilizing polymers, wherein the weight ratio of Compound A, or a pharmaceutically acceptable salt thereof, to one or more stabilizing polymers is between about 5:95 to about 90:10, about 40:60, about 80:20; preferably about 60:40.

Item A2. The amorphous solid dispersion according to item A1, wherein the amorphous solid dispersion is prepared by spray drying, co-grinding, hot-melt extrusion, freeze drying, rotary evaporation, solvent evaporation, co-precipitation, lyophilization, or any suitable solvent removal process. Preferably, the amorphous solid dispersion is made by hot-melt extrusion.

Item A3. The amorphous solid dispersion according to item A1, wherein the amorphous solid dispersion is prepared from Compound A in an amorphous form, a crystalline form, or a mixture thereof.

Item A4. The amorphous solid dispersion according to item A3, wherein the amorphous solid dispersion is prepared from Compound A in a crystalline form.

Item A5. The amorphous solid dispersion according to item A4, wherein the amorphous solid dispersion is prepared from Compound A in an anhydrous crystalline form.

Item A6. The amorphous solid dispersion according to item A5, wherein the amorphous solid dispersion is prepared from anhydrous Form A of Compound A.

Item A7. The amorphous solid dispersion according to item A4, wherein the amorphous solid dispersion is prepared from Compound A in a hydrate crystalline form, e.g., a monohydrate crystalline form.

Item A8. The amorphous solid dispersion according to item A7, wherein the amorphous solid dispersion is prepared from Compound A Monohydrate Form H_{A} of Compound A.

Item A9. The amorphous solid dispersion according to item A1, wherein the one or more stabilizing polymers is selected from the group consisting of polyvinyl pyrrolidone (povidone or PVP), polyvinylpolypyrrolidone (crospovidone or PVP-XL), hydroxypropyl cellulose (HPC), low-substituted hydroxypropyl cellulose (L-HPC), hypromellose (HPMC), hypromellose acetate succinate (HPMC-AS), hypromellose phthalate (HPMC-P), carboxymethyl cellulose, croscarmellose sodium (NaCMC), methyl cellulose, hydroxyethyl cellulose, carboxyethyl cellulose, carboxymethyl cellulose, carboxymethylhydroxyethyl cellulose, polyethylene glycol (PEG), polyvinylalcohol, polyvinylpyrrolidone-vinyl acetate copolymer (copovidone or PVP/VA), polyvinyl alcohol-polyethylene glycol co-polymer, polyvinyl caprolactam-polyvinyl acetate-polyethylene glycol graft copolymer, polyacrylates, polymethacrylates, or a mixture thereof.

Item A10. The amorphous solid dispersion according to item A9, wherein the one or more stabilizing polymers is polyvinyl pyrrolidone (PVP) or polyvinylpolypyrrolidone (crospovidone or PVP XL), preferably poly(vinylpyrrolidone-co-vinyl acetate 60:40 (PVP VA64) or PVP K30.

Item A11. The amorphous solid dispersion according to item A9, wherein the one or more stabilizing polymers is croscarmellose sodium (NaCMC, Ac-Di-Sol) or low-substituted hydroxypropyl cellulose (L-HPC).

Item A12. The amorphous solid dispersion according to item A9, wherein the one or more stabilizing polymers is a polymethacrylate, preferably Eudragit® L100 (methacrylic acid-methyl methacrylate copolymer (1:1)), or Eudragit® L100-55 (poly(methacrylic acid, ethyl acrylate) 1:1).

Item A13. The amorphous solid dispersion according to item A9, wherein the one or more stabilizing polymers is hypromellose (HPMC), preferably HPMC 2910.

Item A14. The amorphous solid dispersion according to item A9, wherein the one or more stabilizing polymers is hypromellose acetate succinate (HPMC-AS), preferably HPMC-AS-L, HPMC-AS-M, or HPMC-AS-H.

Item A15. The amorphous solid dispersion according to item A14, wherein the one or more stabilizing polymers is a mixture of hypromellose (HPMC) and hypromellose acetate succinate (HPMC-AS).

Item A16. The amorphous solid dispersion according to item A1 to A15, optionally further comprising one or more pharmaceutically acceptable excipient(s) selected from solubilzers, diluents, binders, disintegrants, fillers, lubricants, glidants, surfactants, stabilizing agents, antioxidants, alkaline stabilizers, colors, flavors, preservatives, and combinations thereof.

Item A17. The amorphous solid dispersion according to item A1 to A16, further comprising a glidant selected from the group consisting of silicon dioxide, stearic acid, magnesium stearate, calcium stearate, talc, hydrogenated castor oil, sucrose esters of fatty acid, microcrystalline wax, yellow beeswax, white beeswax, and the like and mixtures thereof, preferably wherein the glidant is silicon dioxide, more preferably colloidal silicon dioxide.

Item A18. The amorphous solid dispersion according to item A1 to A16, further comprising a solubilizer selected from the group consisting of polyoxyethylene alkylaryl ethers, polyethylene glycol fatty acid esters, D-α-tocopheryl polyethylene glycol succinate, polyoxyethylene sorbitan fatty acid ester, alkyl sulfates or sulfonates (such as sodium lauryl sulfate or sodium dioctyl sulfosuccinate), lecithin, polyethoxylated castor oils and the like and mixtures thereof.

Item A19. The amorphous solid dispersion according to item A1 to A18, wherein Compound A is present in an amount from about 1% to about 90% (w/w), from about 10% (w/w) to about 85% (w/w), preferably from about 15% (w/w) to about 80% (w/w), from about 20% (w/w) to about 75% (w/w), or from about 30% (w/w) to about 60% (w/w) of the dispersion.

Item A20. The amorphous solid dispersion according to item A1 to A19, wherein the ratio of amount by weight of Compound A and amount by weight of the one or more stabilizing polymers therein of the dispersion is about between 5:95 to 90:10, preferably about 40:60, about 60:40, or about 80:20.

Item A21. The pharmaceutical composition comprising an amorphous solid dispersion according to item A1 to A20 and optionally one or more pharmaceutically acceptable excipient(s) selected from solubilzers, diluents, binders, disintegrants, fillers, lubricants, glidants, surfactants, stabilizing agents, antioxidants, alkaline stabilizers, colors, flavors, preservatives, and combinations thereof.

Item A22. The pharmaceutical composition according to item A21, wherein the pharmaceutical composition is in the form of a tablet, a capsule, a caplet, beads, granules, oral suspension, oral solution, or microemulsion, preferably a tablet.

Item A23. The pharmaceutical composition according to item A21 to A22, wherein the pharmaceutical composition comprises from about 10 mg to about 300 mg of Compound A, preferably 50 mg, 100 mg, 200 mg, or 300 mg of Compound A.

Item A24. The pharmaceutical composition according to claim A21 to A23, wherein the pharmaceutical composition is in the form of a tablet or a capsule comprising: (a) an amorphous solid dispersion of Compound A, wherein the amorphous solid dispersion is in the form of granules, (b) at least one intra-granular excipient, (c) at least one extra-granular excipient, and (d) optionally, a coating.

Item A25. The pharmaceutical composition according to item A24, wherein the extra-granular excipient or excipients comprise a diluent which is selected from the group consisting of microcrystalline cellulose, calcium carbonate, calcium phosphate-dibasic, calcium phosphate-tribasic, calcium sulfate, cellulose powdered, dextrates, dextrins, dextrose excipients, fructose, kaolin, lactitol, lactose, mannitol, sorbitol, starch, starch pregelatinized, sucrose, sugar compressible, sugar confectioners and combinations thereof, preferably wherein the diluent is lactose, microcrystalline cellulose, or a mixture of lactose and microcrystalline cellulose.

Item A26. The pharmaceutical composition according to item A24 to A25, wherein the extra-granular excipients further comprise a disintegrant selected from the group consisting of croscarmellose sodium, low-substituted hydroxypropyl cellulose (L-HPC), polyvinylpolypyrrolidone (crospovidone), sodium bicarbonate, sodium starch glycollate, carboxymethyl cellulose, calcium carboxymethyl cellulose, sodium carboxymethyl cellulose, starch, crystalline cellulose, hydroxypropyl starch, pregelatinized starch, and mixtures thereof, preferably wherein the disintegrant is selected from croscarmellose sodium, sodium bicarbonate and crospovidone, more preferably wherein the disintegrant is croscarmellose sodium.

Item A27. A method for preparing a pharmaceutical composition according to item A21 to A24, which comprises the steps of: mixing Compound A, or a pharmaceutically acceptable salt thereof, or an amorphous form thereof, or a crystalline form thereof, with one or more stabilizing polymers, and optionally one or more pharmaceutically acceptable excipients; heating the mixture to form a molten mass; extruding the molten mass; cooling the molten mass to form an amorphous solid dispersion, and optionally granulating the amorphous solid dispersion and/or optionally compacting the amorphous solid dispersion or granules of the amorphous solid dispersion for further processing with one or more pharmaceutically acceptable excipients to form a composition suitable for use in dosage forms such as tablets and capsules. Preferably, the amorphous solid dispersion is milled to form granules.

Item A28. The pharmaceutical composition according to any one of items A21 to A26 for use as a medicament.

Item A29. The pharmaceutical composition according to any one of items A21 to A26, for use in the treatment of cancer.

Item A30. The pharmaceutical composition according to any one of items A21 to A26, for use in the treatment of cancer, in particular in the treatment of cancers harboring MAPK pathway alterations such as KRAS-mutant NSCLC (non-small cell lung cancer), KRAS-mutant pancreatic cancer (e.g., KRAS-mutant pancreatic ductal adenocarcinoma (PDAC)), KRAS-mutant CRC (colorectal cancer), and NRAS-mutant melanoma.

Item A31. A method of treating cancer comprising administering to a subject in need thereof a therapeutically effective amount of the pharmaceutical composition according to any one of items A21 to A26.

Item A32. The method of A30, wherein the cancer is harboring MAPK pathway alterations such as KRAS-mutant NSCLC (non-small cell lung cancer), KRAS-mutant pancreatic cancer (e.g., KRAS-mutant pancreatic ductal adenocarcinoma (PDAC)), KRAS-mutant CRC (colorectal cancer), and NRAS-mutant melanoma.

### DETAILED DESCRIPTION OF THE INVENTION

As used herein the term “Compound A” refers to N-(3-(2-(2-hydroxyethoxy)-6-morpholinopyridin-4-yl)-4-methylphenyl)-2-(trifluoromethyl)isonicotinamide, or a pharmaceutically acceptable salt thereof.

As used herein, and unless context clearly indicates, the term “Compound A” refers to N-(3-(2-(2-hydroxyethoxy)-6-morpholinopyridin-4-yl)-4-methylphenyl)-2-(trifluoromethyl)isonicotinamide as the free base. Reference to the “free base” of Compound A or the “free form” of Compound A means that Compound A is present as the free base and not as a salt of Compound A.

In certain aspects, an amorphous form of the free base of Compound A, a crystalline form of the free base of Compound A, a mixture of the amorphous form and a crystalline form, of Compound A may be used in the preparation of amorphous solid dispersion formulations of Compound A of the present invention.

As used herein, the term “amorphous” refers to a solid form of a compound that is not substantially crystalline. An amorphous compound possesses no long-range order and does not display a definitive X-ray diffraction pattern with reflections.

In an embodiment, the amorphous solid dispersion is prepared from Compound A which is in a crystalline form. In one embodiment, the crystalline form of Compound A which is used in the preparation of the amorphous solid dispersion of the invention is the crystalline anhydrate Form A.

Anhydrate Form A is referred to as “Form A” and characterized in WO/2020/230028, which is hereby incorporated in its entirety. It can be prepared as described in Example 2 of WO/2020/230028.

Anhydrate Form A of Compound A exhibits an X-ray powder diffraction pattern having at least one, two or three characteristic peaks expressed in degrees 2-Theta (°2θ) at angles of 5.8°+/−0.2°, 11.70+/−0.2° and 14.8°+/−0.2° when measured using CuKα radiation. In another embodiment, the polymorph Form A exhibits at least one, two or three characteristic peaks at angles of 5.8°+/−0.2°, 11.7°+/−0.2, 14.8°+/−0.2°, 15.2°+/−0.2° and 18.7°+/−0.2° when measured using CuKα radiation. In another embodiment, the polymorph Form A exhibits at least one, two, three, four or five characteristic peaks at angles of 5.8°+/−0.2°, 10.0°+/−0.2°, 11.7°+/−0.2°, 12.6°+/−0.2°, 13.1°+/−0.2°, 14.8°+/−0.2°, 15.2°+/−0.2°, 18.7°+/−0.2°, 20.2°+/−0.2° and 25.1°+/−0.2° when measured using CuKα radiation.

In another embodiment, the crystalline form of Compound A (free base) is the crystalline Monohydrate Form H_{A} of Compound A.

The crystalline Monohydrate Form H_{A} of Compound A is described in WO/2020/230028, which is hereby incorporated in its entirety, and may be prepared according to the procedure described in Example 8 of WO/2020/230028. In one embodiment, the Monohydrate Form H_{A} exhibits an X-ray powder diffraction pattern having at least one, two or three characteristic peaks expressed in degrees 2-Theta (°2θ) at angles of 7.3°+/−0.2°, 10.7°+/−0.2° and 23.0°+/−0.2° when measured using CuKα radiation. In another embodiment, the Monohydrate Form H_{A} exhibits at least one, two or three characteristic peaks at angles of 7.3°+/−0.2°, 10.7°+/−0.2°, 16.3°+/−0.2°, 16.7°+/−0.2° and 23.0°+/−0.2° when measured using CuKα radiation. In another embodiment, the Monohydrate Form H_{A} exhibits at least one, two, three, four or five characteristic peaks at angles of 7.3°+/−0.2°, 10.7°+/−0.2°, 16.3°+/−0.20, 16.70+/−0.2°, 17.40+/−0.2°, 23.00+/−0.2°, 24.30+/−0.2°, 25.30+/−0.2°, 28.30+/−0.2° and 32.00+/−0.2° when measured using CuKα radiation.

The crystalline Monohydrate Form H_{A} of Compound A can be characterized by having an X-ray powder diffraction pattern with at least one, two, three, four or five peaks having an angle of refraction 2 theta (θ) values selected from 7.3, 10.7, 16.3, 16.7, 17.4, 23.0, 24.3, 25.3, 28.3, 32.0 when measured using CuKα radiation, wherein said values are plus or minus 0.2° 20. The crystalline Monohydrate Form H_{A} of Compound A can also be characterized by having a differential scanning calorimetry curve comprising an endothermic event from about 35° C. to 135° C. and shows an onset of dehydration at about 94° C. The crystalline Monohydrate Form H_{A} of Compound A can also be characterized by having a thermogravimetric analysis curve showing a mass loss of not more than 3.7 weight % between about 43° C. to 135° C., when heated from 30° C. to 300° C. at a rate of 20° C./min.

The use of Monohydrate H_{A} of Compound A to prepare the amorphous solid dispersions of the present invention led to oral dosage forms with a higher drug loading than possible with other solid forms not using Monohydrate H_{A} of Compound A, e.g., anhydrate H_{A} of Compound A as the starting material.

The term “pharmaceutically acceptable” refers to those compounds, materials, compositions, and/or dosage forms which are suitable for use in contact with the tissues of human beings and animals without excessive toxicity, irritation, allergic response, other problem or complication, commensurate with a reasonable benefit/risk ratio.

The terms “pharmaceutical composition,” “pharmaceutical product,” “pharmaceutical dosage form,” “dosage form,” “pharmaceutical formulation,” etc., refer to a pharmaceutical composition that may be administered to a patient in need of treatment, which may be in any conventional formulation, e.g., in the form of a powder, a granule, a pill, a capsule, a tablet, a solution, a suspension, or a patch, etc.

The term solid dispersion in general means a system in solid state comprising at least two components, wherein one component is dispersed substantially evenly throughout the other component(s). For example, solid dispersions may be the dispersion of one or more active ingredients in an inert carrier or matrix at solid state, prepared by the melting, solvent, or melting-solvent methods. While not wishing to be bound by theory, in a solid dispersion, the drug may be present in a molecular state, colloidal state, metastable state, or an amorphous state. Formation of a molecular dispersion may provide a means of reducing the drug particle size to nearly molecular levels (i.e., there are no particles). As the polymer dissolves, the drug is exposed to the dissolution media moleculary or as fine particles, which are amorphous, and which can dissolve and be absorbed more rapidly than larger crystalline particles.

The term “solid dispersion” refers to a dispersion of a compound, particularly a drug substance or active pharmaceutical ingredient (API), within a polymer or carrier.

The term “amorphous solid dispersion” refers to a substantially non-crystalline molecular dispersion of a compound, particularly a drug substance or API, within a polymer or carrier. The compound may be in an amorphous form, a crystalline form, or a mixture prior to preparation of the solid dispersion.

An amorphous solid dispersion where the drug substance is dispersed using one or more polymers is also known as a polymer-stabilized amorphous solid dispersion (PSASD). A PSASD formulation is a thermodynamically unstable solid state system, in which one or more active ingredients are dispersed substantially evenly throughout the other components of the formulation, and is stabilized using one or more polymers. In an embodiment, the amorphous solid dispersions of the present invention may be prepared from Compound A in a crystalline form.

In an embodiment, the amorphous solid dispersion of the present invention may be prepared from Compound A in an anhydrous crystalline form.

In an embodiment, the amorphous solid dispersion of the present invention may be prepared from Compound A in the anhydrous crystalline Form A.

In an embodiment, the amorphous solid dispersion of the present invention may be prepared from Compound A in the monohydrate crystalline form.

In an embodiment, the amorphous solid dispersion of the present invention may be prepared from Compound A in the monohydrate crystalline form Monohydrate Form H_{A}.

### Methods for Making Solid Dispersions

It has been found that solid dispersion formulations according to the invention are useful for improving bioavailability by increasing solubility of active agents with low solubility such as Compound A.

Amorphous solid dispersions are high energy formulations which present additional challenges since they are, by nature, thermodynamically unstable. Consequently, their successful development depends in good measure on the understanding of the specific interactions responsible for their stabilization (Serajuddin, A. T. M. J. Pharm. Sci. 1999, 88, 1058-1066; Janssens, S. and Van den Mooter, G. J. Pharm. Phamacol. 2009, 61, 1571-1586.). However, there are no universal or reliable methods to select either a technology or a polymer to have guaranteed amorphous stability and improved bioavailability. Solubility parameters have been reported to aid the selection of the polymers. However, there is generally no method of predicting the benefit of using one particular polymer and/or one particular method for preparing solid dispersion over another in terms of providing a stable amorphous dispersion of a given drug.

An added unknown is also the effect of the drug loading of a given pharmaceutical formulation. The drug loading in amorphous solid dispersions has also been found to be critical to the stability of any given formulation. In general, the lower the drug load, the better the stability of the dispersion. Above a certain drug loading, the amorphous solid dispersion poses a high risk in re-crystallization during shelf-life storage and therefore diminishes the benefit of the improved solubility and bioavailability. It can thus be seen that although, in principle, amorphous solid dispersions may in theory improve the bioavailability of a drug substance, providing a stable pharmaceutical dosage form of a drug substance in the form of an amorphous solid dispersion is not a trivial exercise.

In spite of these hurdles, the present invention provides an amorphous solid dispersion comprising Compound A, as the free base or as a pharmaceutically acceptable salt thereof, and one or more stabilizing polymers, wherein Compound A may be successfully administered to a patient in need thereof in a manner which is bioavailable and wherein the oral dosage form of Compound A is stable.

The amorphous solid dispersions of the present invention may be formed by any conventional technique, e.g., spray drying, co-grinding, hot-melt extrusion, freeze drying, rotary evaporation, solvent evaporation, co-precipitation, lyophilization, or any suitable solvent removal process.

While not wishing to be bound by theory, the stabilizing polymer in the solid dispersion may reduce the molecular mobility of the drug to avoid the phase separation and re-crystallization of drug during storage. However, it would be appreciated that the presence of certain extraneous excipients may compromise the stability of the solid dispersion (e.g., to remain amorphous). The polymer and process selection for amorphous solid dispersions have been found to play critical role on solubility and stabilizing the solid dispersions. However, there is no absolute method a priori to judge whether a given polymer or process will provide adequate solubility and stability of the amorphous solid dispersions.

In an embodiment, the amorphous solid dispersion of the present application comprises Compound A and one or more stabilizing polymers, wherein the one or more stabilizing polymers is selected from the group consisting of polyvinyl pyrrolidone (povidone or PVP), polyvinylpolypyrrolidone (crospovidone or PVP-XL), hydroxypropyl cellulose (HPC), low-substituted hydroxypropyl cellulose (L-HPC), hypromellose (HPMC), hypromellose acetate succinate (HPMC-AS), hypromellose phthalate (HPMC-P), carboxymethyl cellulose, croscarmellose sodium (NaCMC), methyl cellulose, hydroxyethyl cellulose, carboxyethyl cellulose, carboxymethyl cellulose, carboxymethylhydroxyethyl cellulose, polyethylene glycol (PEG), polyvinylalcohol, polyvinylpyrrolidone-vinyl acetate copolymer (copovidone or PVP/VA), polyvinyl alcohol-polyethylene glycol co-polymer, polyvinyl caprolactam-polyvinyl acetate-polyethylene glycol graft copolymer, polyacrylates, polymethacrylates, or a mixture thereof.

In an embodiment, the amorphous solid dispersion of the present application comprises Compound A and one or more stabilizing polymers, wherein the one or more stabilizing polymers is polyvinyl pyrrolidone (PVP), Various specific molecular grades of PVP may be used; for example poly(vinylpyrrolidone-co-vinyl acetate 60:40 (PVP VA64) or PVP K30.

In an embodiment, the amorphous solid dispersion of the present application comprises Compound A and one or more stabilizing polymers, wherein the one or more stabilizing polymers is polyvinylpolypyrrolidone (crospovidone or PVP XL).

In an embodiment, the amorphous solid dispersion of the present application comprises Compound A and one or more stabilizing polymers, wherein the one or more stabilizing polymers is croscarmellose sodium (NaCMC) or low-substituted hydroxypropyl cellulose (L-HPC).

In an embodiment, the amorphous solid dispersion of the present application comprises Compound A and one or more stabilizing polymers, wherein the one or more stabilizing polymers is a polymethacrylate, preferably Eudragit® L100, or Eudragit® L100-55.

Eudragit® is the brand name for a diverse range of polymethacrylate-based copolymers. It includes anionic, cationic, and neutral copolymers based on methacrylic acid and methacrylic/acrylic esters or their derivatives. Eudragit® L100 is an anionic copolymer of methacrylic acid and methyl methacrylate with ratio of the free carboxyl groups to the ester groups of approximately 1:1. Eudragit® L 100-55 is an anionic copolymer based on methacrylic acid and ethylacrylate where the ratio of free carboxyl groups to the ester groups is approximately 1:1.

In a preferred embodiment, the amorphous solid dispersion of the present application comprises Compound A, one or more stabilizing polymers, wherein the one or more stabilizing polymers is hypromellose (HPMC). Various grades of hypromellose e.g containing varying ratios of hydroxypropyl and methoxy groups may be used. The following hypromellose types, as specified in the Pharmacopeia (Ph. Eur., USP/NF and JP) may be used.

TABLE 1
H. types specified in Ph. Eur., USP/NF and JP

| Substitution type | Methoxy groups [%] | Hydroxypropoxy groups [%] |
|---|---|---|
| HPMC 1828 | 16.5 to 20.0 | 23.0 to 32.0 |
| HPMC 2208 | 19.0 to 24.0 | 4.0 to 12.0 |
| HPMC 2906 | 27.0 to 30.0 | 4.0 to 7.5 |
| HPMC 2910 | 28.0 to 30.0 | 7.0 to 12.0 |

An example of a stabilizing polymer to be used in the invention is HPMC 2910, which has about 29% methoxy groups and about 10% hydroxypropoxy groups. HPMC 2910 is also known by the name “HPMC 603”.

In an embodiment, the amorphous solid dispersion of the present application comprises Compound A, one or more stabilizing polymers, wherein the one or more stabilizing polymers is hypromellose acetate succinate (HPMC-AS), preferably HPMC-AS-L, HPMC-AS-M, or HPMC-AS-H.

In an embodiment, the amorphous solid dispersion of the present application comprises Compound A, one or more stabilizing polymers, wherein the one or more stabilizing polymers is a mixture of hypromellose (HPMC) and hypromellose acetate succinate (HPMC-AS).

The amorphous solid dispersions of the present invention may further comprise optionally one or more pharmaceutically acceptable excipients selected from solubilizers, diluents, binders, disintegrants, fillers, lubricants, glidants, surfactants, stabilizing agents, antioxidants, alkaline stabilizers, colors, flavors, preservatives, and combinations thereof.

As used herein, term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. Examples of such excipients include, but are not limited to, solubilizers, diluents, binders, disintegrants, fillers, lubricants, glidants, surfactants, stabilizing agents, antioxidants, alkaline stabilizers, colors, flavors, and preservatives. One of ordinary skill in the art may select one or more of the aforementioned excipients with respect to the particular desired properties of the solid oral dosage form by routine experimentation and without any undue burden. The amount of each excipient used may vary within ranges conventional in the art. The following references which are all hereby incorporated by reference discloses techniques and excipients used to formulate oral dosage forms. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The amorphous solid dispersions of the present invention may optionally include one or more lubricants or glidants, i.e., substance or a material that improves the properties of the solid dispersion, e.g., processability. Suitable lubricants or glidants for use in compositions of the present invention include silicon dioxide, stearic acid, magnesium stearate, calcium stearate, talc, hydrogenated castor oil, sucrose esters of fatty acid, microcrystalline wax, yellow beeswax, white beeswax, and the like and mixtures thereof, preferably silicon dioxide, more preferably colloidal silicon dioxide.

In an embodiment, the amorphous solid dispersion of the present application comprises Compound A, one or more stabilizing polymers, and optionally one or more pharmaceutically acceptable excipients that is a glidant. In particular, it has been found that when a mixture of a glidant such as silicon dioxide, a stabilizing polymer (e.g., HPMC) and Compound A is blended and subjected to a hot-melt extrusion process, the resulting extrudate showed improved milling attributes, better compressibility profiles and gave an improvement in disintegration times of the resulting oral dosage forms.

The amorphous solid dispersions of the present invention may optionally include one or more solubilizers, i.e., additives which increase solubility or dissolution rate of the pharmaceutical active ingredient in the solid dispersion or additives which act as pore-forming agents in the solid dispersion. The solubilizers can be selected from surfactants, non-ionic co-polymers, bile salt, and hydrotropes. Suitable solubilizers for use in compositions of the present invention include, but not limited to, cyclodextrins, poloxamers, polyvinylalcohol, polyvinylpyrrolidone, polyoxyethylene sorbitan fatty acid esters such as polysorbate 80, alkyl sulfates or sulfonates such as sodium lauryl sulfate or sodium dioctyl sulfosuccinate, lecithins, D-α-tocopheryl polyethylene glycol succinate, polyethoxylated castor oils such as Cremophor® RH 40 and Cremophor® EL/ELP, polyoxyethylene stearate, polymethacrylate-based copolymers such as Eudragit® EPO and Eudragit® L 100-55, hypromellose acetate succinate, hydroxypropyl methylcellulose, hydroxypropyl cellulose, polyvinylpyrrolidone-vinyl acetate copolymer, polyvinyl caprolactam-polyvinyl acetate-polyethylene glycol graft copolymer such as Soluplus®, polyoxyethylene alkylaryl ethers such as polyoxyethylene stearyl ether, polyethylene glycol fatty acid esters such as PEG stearate or PEG hydroxy stearate, sodium taurocholate, sodium benzoate, and the like and combinations thereof.

The amorphous solid dispersions of the present invention may optionally include one or more surfactants. Surfactants are compounds which are capable of improving the wetting of the drug and/or enhancing the dissolution. The surfactants can be selected from hydrophilic surfactants or lipophilic surfactants or mixtures thereof. The surfactants can be anionic, nonionic, cationic, and zwitterionic surfactants. Surfactants according to the present invention may include, but not limited to, nonionic copolymer such as Poloxamer 188, polyoxyethylene alkylaryl ethers such as polyoxyethylene lauryl ether, polyoxyethylene cetyl ether, polyoxyethylene stearyl ether; polyethylene glycol fatty acid esters such as PEG monolaurate, PEG dilaurate, PEG distearate, PEG dioleate, PEG stearate, PEG hydroxy stearate; vitamin E PEG 1000 succinate; polyoxyethylene sorbitan fatty acid ester such as polysorbate 40, polysorbate 60, polysorbate 80; sorbitan fatty acid mono esters such as sorbitan monolaurate, sorbitan monooleate, sorbitan sesquioleate, sorbitan trioleate, alkyl sulfates or sulfonates such as sodium lauryl sulfate, sodium dioctyl sulfosuccinate, lecithin, stearylic alcohol, cetostearylic alcohol, cholesterol, polyoxyethylene ricin oil, polyoxyethylene fatty acid glycerides, Cremophor® RH 40, cremophor EL/ELP, and the like or combinations thereof.

In some aspects, the percentage drug loading of Compound A in the amorphous solid dispersion is from about 1% to about 90% (w/w) (e.g., from 1% to 5%, from 5% to 10%, from 5% to 20%, from 5% to 30%, from 5% to 40%, from 5% to 50%, from 5% to 60%, from 5% to 70%, from 5% to 80%, from 5% to 90%, from 10% to 20%, from 10% to 30%, from 10% to 40%, from 10% to 50%, from 10% to 60%, from 10% to 70%, from 10% to 80%, from 10% to 90%, from 20% to 30%, from 20% to 40%, from 20% to 50%, from 20% to 60%, from 20% to 70%, from 20% to 80%, from 20% to 90%, from 21% to 30%, from 21% to 34%, from 21% to 40%, from 21% to 50%, from 21% to 60%, from 21% to 70%, from 21% to 80%, from 21% to 90%, from 30% to 40%, from 30% to 50%, from 30% to 60%, from 30% to 70%, from 30% to 80%, from 30% to 90%, from 36% to 40%, from 36% to 49%, from 36% to 60%, from 36% to 70%, from 36% to 80%, from 36% to 90%, from 40% to 50%, from 40% to 60%, from 40% to 70%, from 40% to 80%, from 40% to 90%, from 50% to 60%, from 50% to 70%, from 50% to 80%, from 50% to 90%, 51% to 60%, from 51% to 70%, from 51% to 80%, from 51% to 90%, from 60% to 70%, from 60% to 80%, from 60% to 90%, from 70% to 80%, and from 70% to 90%). In some preferred embodiments, the percentage loading of Compound A is from about 1% to about 90% (w/w), from about 10% (w/w) to about 85% (w/w), preferably from about 15% (w/w) to about 80% (w/w), from about 20% (w/w) to about 75% (w/w), or from about 30% (w/w) to about 60% (w/w).

In some aspects, the amorphous solid dispersion of the present invention has a ratio of amount by weight of Compound A and amount by weight of the one or more stabilizing polymers is between about 5:95 to about 90:10, about 40:60, about 80:20; preferably about 60:40.

In an aspect, methods are provided for preparing amorphous solid dispersions as described herein, which comprise preparing a mixture (e.g., a solid mixture) of Compound A, one or more stabilizing polymers, and optionally one or more pharmaceutically acceptable excipients such as a glidant, heating the mixture to form a molten mass; extruding the molten mass; cooling the molten mass to form an amorphous solid dispersion (e.g., a hot-melt extrusion).

The resultant amorphous solid dispersion is directly processed into final dosage forms or further processed into final dosage forms. For example, the amorphous solid dispersion can be blended with one or more excipients, as described herein, after being milled, granulated and then compacted to produce a final blend for encapsulating or tableting. In particular embodiments, the solid dispersion may be combined with one or more excipient(s) e.g., such as a binding agent, a filler, a disintegrating agent, a wetting agent, a glidant, and a lubricant and the resulting mixture may be granulated to form granules comprising the solid dispersion and one or more excipients.

### Hot-Melt Extrusion Process

In certain aspects, solid dispersions of the invention may be made by hot-melt extrusion (“hot melt extrusion”), e.g., a process whereby a composition is heated and/or compressed to a molten (or softened) state and subsequently forced through an orifice in a die where the extruded product is formed into its final shape in which it solidifies upon cooling. Hot-melt extrusion is simple and easy to operate, and decreases energy consumption, and increases productivity.

In the hot melt extrusion process, a blend is conveyed through one or more heating zones typically by a screw mechanism. The screw or screws are rotated by a variable speed motor inside a cylindrical barrel where only a small gap exists between the outside diameter of the screw and the inside diameter of the barrel. In this conformation, high shear is created at the barrel wall and between the screw fights by which the various components of the powder blend are well mixed and disaggregated. The die can be a dual manifold, multi-manifold or feed-block style die. As used herein, the term extrudate refers to hot-melt extruded composition.

In an embodiment, amorphous solid dispersion of the present application is obtained by hot-melt extrusion. A physical mixture of Compound A, one or more stabilizing polymers, and optionally one or more pharmaceutically acceptable excipients may be subjected to hot-melt extrusion from about 25° C. to about 200° C., e.g., from about 25° C. to about 170° C. through a hot-melt extruder (such as Thermo Fisher Pharma 11 mm twin screw or Leistritz ZSE 18 mm HPe-PH twin screw) having twin screws. The obtained hot-melt extrusion product may be chilled, milled and passed through a 0.5 mm screen.

In other embodiments, the mixture may be fed into a hot-melt extruder having temperature zones between about 25° C. to about 200° C., e.g., between about 25° C. to about 170° C. to produce an extrudate.

Preferably, the hot-melt extrusion is to be carried out at a temperature that allows the melting of Compound A and one or more stabilizing polymers. In certain embodiments, the mixture of Compound A and one or more stabilizing polymers may be heated near or past the glass transition temperature T_{g} or melting temperature Tₘ to form a liquid mixture. After the mixture is heated to form a molten mass, it may be extruded and cooled to form a solid dispersion.

The temperature and screw speed of the hot-melt extruder may be selected based on the type of pharmaceutically acceptable carrier employed, e.g., to extrude the target mixture smoothly, wherein the extrusion speed and the yield meet desired requirements and desired amorphization and dispersion effect.

In certain aspects, optionally, a glidant may also be included in the mixture of Compound A and one or more stabilizing polymers to enhance milling attributes of extrudate, compressibility profile, and improve the disintegration time. An exemplary glidant includes silicon dioxide, in any useful or effective amount (e.g., from about 1% to about 10% (w/w), e.g., about 3% (w/w)) of the amorphous solid dispersion.

The extrudates can optionally be pelletized or milled to form a solid dispersion amenable for further processing in a suitable unit dosage form. In certain aspects, the extrudate is then pelletized and milled to produce granules of the extrudate. The milled/pelletized extrudate can be used for encapsulating or tableting. In particular embodiments, the milled/pelletized extrudate form an internal phase (e.g., granular component) that can be sieved and blended with various pharmaceutically acceptable excipients, such as a binding agent, a filler, a disintegrating agent, a wetting agent, a glidant, and a lubricant that form an external phase (e.g., extra-granular component), where the resultant blend is used for encapsulating or tableting.

### Pharmaceutical Compositions

The amorphous solid dispersions of the present invention may be used for filling any one of the unit dosage forms described herein (e.g., a capsule) or for tableting.

The solid dispersion can optionally be further processed before filling or tableting. Exemplary further processing includes spheronizing, pelletizing, milling, injection molding, sieving, and/or calendering the solid dispersion.

The amorphous solid dispersions of the present invention can be optionally subjected to a particle size reduction procedure before or after the completion of drying or cooling of the product to produce desired particle sizes and particle size distributions. Milling or micronization can be performed to achieve the desired particle sizes or distributions. Equipment that may be used for particle size reduction include, without limitation thereto, ball mills, roller mills, hammer mills, pin mills, and jet mills. Preferably, the amorphous solid dispersion of the present invention is milled to form granules.

The granules of the amorphous solid dispersions of the present invention may be combined with one or more pharmaceutically acceptable excipients to make other pharmaceutical compositions, or a finished dosage form. The one or more additional pharmaceutically acceptable excipients can be selected from solubilizers, diluents, binders, disintegrants, fillers, lubricants, glidants, surfactants, stabilizing agents, antioxidants, alkaline stabilizers, colors, flavors, preservatives, and combinations thereof.

In an embodiment, the pharmaceutical composition of the present invention comprises an amorphous solid dispersion and optionally one or more pharmaceutically acceptable excipients selected from solubilzers, diluents, binders, disintegrants, fillers, lubricants, glidants, surfactants, stabilizing agents, antioxidants, alkaline stabilizers, colors, flavors, preservatives, and combinations thereof.

The pharmaceutical compositions of the present invention may be in the form of an oral dosage form such as a tablet, a capsule, a caplet, beads, granules, oral suspension, oral solution, or microemulsion, preferably a tablet.

The tablets or granules of the present invention can be uncoated or coated by known techniques to delay disintegration and absorption in the gastrointestinal tract and thereby provide a sustained action over a longer period. For example, tablets can be coated with a suitable polymer or a conventional coating material to achieve, for example, greater stability in the gastrointestinal tract, or to achieve the desired rate of release, for example the tablet can be coated with hypromellose (HPMC), magnesium stearate, polyethylene glycol (PEG), polyvinyl alcohol (PVA), Opadry®, Opadry II®, or mixtures thereof. For example, a time delay material such as glyceryl monostearate or glyceryl distearate can be employed. Tablets of any shape or size can be prepared, and they can be opaque, coloured, or flavoured. Specifically, the pharmaceutical composition as disclosed herein, is in the form of a filmed coated tablet.

In an embodiment, a pharmaceutical composition of the present invention comprises granules of the amorphous solid dispersion of Compound A that is optionally mixed with one or more additional pharmaceutically acceptable excipients (e.g., extra-granular material) and either compressed into tablets or filled into hard gelatin capsules.

In an embodiment, the pharmaceutical composition of the present invention is in a form of a tablet or a capsule comprising: (a) an amorphous solid dispersion of Compound A in the form of granules, (b) at least one intra-granular excipient, (c) at least one extra-granular excipient, and (d) optionally, a coating.

The extra-granular excipients may be selected from one, or more, or all of (i) a diluent, (ii) a disintegrant; (iii) a lubricant and (iv) a glidant.

The diluent may be present from about 10 to about 60% weight by weight (w/w) of the total composition.

The disintegrant may be present from about 1 to about 10% weight by weight (w/w) of the total composition.

The lubricant may be present from about 1 to about 2% by weight by weight (w/w) of the total composition.

The glidant may be present from about 1 to about 3% weight by weight (w/w) of the total composition.

The extra-granular excipients may also be selected from one, or more, or all of (i) a diluent, such as microcrystalline cellulose, lactose, or a combination thereof; (ii) a disintegrant such as croscopovidone, croscarmellose sodium, or a combination thereof, (iii) a lubricant (e.g., sodium stearyl fumarate) and (iv) a glidant such as silicon dioxide.

The extra-granular excipients may be selected from one, or more, or all of (i) 10-60% of a diluent, such as microcrystalline cellulose, lactose, or a combination thereof, (ii) 1-10% of a disintegrant such as croscopovidone, croscarmellose sodium, or a combination thereof; (iii) 1-2% of a lubricant (e.g., sodium stearyl fumarate) and (iv) 1-3% of a glidant such as silcon dioxide, where the % refers to the % weight by weight (w/w) of the total composition.

The invention provides a pharmaceutical composition comprising granules of an amorphous solid dispersion as described herein and an extra-granular phase.

The pharmaceutical compositions of the present invention may include one or more lubricants or glidants. In an embodiment, suitable lubricants or glidants include silicon dioxide, stearic acid, magnesium stearate, sodium stearyl fumarate, calcium stearate, talc, hydrogenated castor oil, sucrose esters of fatty acid, microcrystalline wax, yellow beeswax, white beeswax, and the like and mixtures thereof.

In an embodiment, a glidant is included either in intra-granular material or extra-granular material or both. Preferably, the glidant is silicon dioxide, more preferably colloidal silicon dioxide.

In an embodiment, the concentration of glidant ranges from about 1% to about 3% w/w of total composition.

In an embodiment, the concentration of lubricant ranges from about 1% to about 2% w/w of total composition. Preferably, the lubricant is magnesium stearate.

The pharmaceutical compositions of the present invention may include one or more disintegrants (e.g., substances or materials added to oral solid dosage forms, e.g., tablet, to aid in their disaggregation, by causing a rapid break-up of solids dosage forms when they come into contact with moisture).

In an embodiment, suitable disintegrating agents include croscarmellose sodium, low-substituted hydroxypropyl cellulose (L-HPC), polyvinylpolypyrrolidone (crospovidone), sodium bicarbonate, sodium starch glycollate, carboxymethyl cellulose, calcium carboxymethyl cellulose, sodium carboxymethyl cellulose, starch, crystalline cellulose, hydroxypropyl starch, pregelatinized starch, and the like and mixtures thereof, preferably sodium bicarbonate and crospovidone, more preferably croscarmellose sodium.

In an embodiment, the disintegrating agent concentration ranges from about 1% to about 10% w/w of total composition.

The pharmaceutical compositions of the present invention may include one or more fillers. In an embodiment, suitable fillers include microcrystalline cellulose, calcium carbonate, calcium phosphate-dibasic, calcium phosphate-tribasic, calcium sulfate, cellulose powdered, dextrates, dextrins, dextrose excipients, fructose, kaolin, lactitol, lactose, mannitol, sorbitol, starch, starch pregelatinized, sucrose, sugar compressible, sugar confectioners and the like and mixtures thereof.

In an embodiment, the concentration of fillers ranges from about 15% to about 60% w/w of total composition, preferably about 10% to about 40%, more preferably about 37% w/w.

The pharmaceutical compositions of the present invention may include one or more diluents. In an embodiment, suitable diluents include microcrystalline cellulose, calcium carbonate, calcium phosphate-dibasic, calcium phosphate-tribasic, calcium sulfate, cellulose powdered, dextrates, dextrins, dextrose excipients, fructose, kaolin, lactitol, lactose, mannitol, sorbitol, starch, starch pregelatinized, sucrose, sugar compressible, sugar confectioners and the like and mixtures thereof, preferably lactose, microcrystalline cellulose, or lactose and microcrystalline cellulose.

In an embodiment, the concentration of diluents ranges from about 15% to about 60% w/w of total composition, preferably about 10% to about 40%, more preferably about 37% w/w.

### Dosage and Administration

The pharmaceutical compositions as described herein may be used in methods of treatment, in which an effective amount of Compound A is administered to a patient. The pharmaceutical compositions described herein may be used to treat cancer, in particular in the treatment of cancers harboring MAPK pathway alterations such as KRAS-mutant NSCLC (non-small cell lung cancer), KRAS-mutant pancreatic cancer (e.g., KRAS-mutant pancreatic ductal adenocarcinoma (PDAC)), KRAS-mutant CRC (colorectal cancer), and NRAS-mutant melanoma.

For administration to animal or human subjects, the pharmaceutical compositions comprise an effective dosage amount of Compound A. The formulation may be prepared using conventional methods, for example, depending on the subject to be treated, the mode of administration, and the type of treatment desired (e.g., prevention, prophylaxis, or therapy).

Compound A may be present in amounts totalling 1-90% by weight of the total weight of the composition.

Preferably, the pharmaceutical composition will be provided in a dosage form that is suitable for oral administration, including but not limited to hard capsules (e.g., hard gelatin capsules or hard hydroxypropyl methylcellulose capsules), soft gelatin capsules, tablets, caplets, enteric coated tablets, chewable tablets, enteric coated hard gelatin capsules, enteric coated soft gelatin capsules, minicapsules, lozenges, films, strips, gelcaps, dragees, suspensions, syrups, or sprinkles. The compositions may be formulated according to conventional pharmaceutical practice.

The dosage levels can be dependent on the nature of the condition, drug efficacy, the condition of the patient, the judgment of the practitioner, and the frequency and mode of administration. The unit dosage forms can be administered to achieve any daily amount described herein, such as by administering one to four times daily (e.g., one, two, three, or four, times daily).

In an aspect, the invention provides a pharmaceutical composition in unit dosage form for oral administration, the composition including from about 10 mg to about 1200 mg (e.g., about 50 mg, about 100 mg, about 150 mg, about 200 mg, about 250 mg, about 300 mg, about 350 mg, about 400 mg, about 450 mg, about 500 mg about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg or about 1200 mg) of Compound A. Preferred dosage amounts include 50 mg, 100 mg, 200 mg, or 300 mg of Compound A.

The term “unit dosage form” refers to a physically discrete unit suitable as a unitary dosage, such as a tablet, caplet, hard capsule, or soft capsule, each unit containing a predetermined quantity of a drug.

By “effective” amount is meant the amount of a drug sufficient to treat, prevent, or ameliorate a condition in a subject or patient. The effective amount of Compound A used to practice the present invention for therapeutic management of a condition may be determined and adjusted by a person of ordinary skill to provide the appropriate amount and dosage regimen, e.g., depending upon one or more of the manner of administration, the age, body weight, sex, and/or general health of the patient.

The term “treat”, treating” or “treatment” of any disease or disorder refers to ameliorating the disease or disorder (e.g., slowing, arresting or reducing the development of the disease, or at least one of the clinical symptoms thereof). In addition those terms refer to alleviating or ameliorating at least one physical parameter including those which may not be discernible by the patient and also to modulating the disease or disorder, either physically (e.g., stabilization of a discernible symptom), physiologically (e.g., stabilization of a physical parameter), or both.

The term “prevent”, “preventing” or “prevention” of any disease or disorder refers to delaying the onset, or development, or progression of the disease or disorder.

The term “about”, as used herein, is intended to provide flexibility to a numerical range endpoint, providing that a given value may be “a little above” or “a little below” the endpoint accounting for variations one might see in the measurements taken among different instruments, samples, and sample preparations. The term usually means within 10%, preferably within 5%, and more preferably within 1% of a given value or range.

The terms “pharmaceutical composition” or “formulation” can be used herein interchangeably, and relate to a physical mixture containing a therapeutic compound to be administered to a mammal, e.g., a human, in order to prevent, treat, or control a particular disease or condition affecting a mammal. The terms also encompass, for example, an intimate physical mixture formed at high temperature and pressure.

The term “oral administration” represents any method of administration in which a therapeutic compound can be administered through the oral route by swallowing, chewing, or sucking an oral dosage form. Such oral dosage forms are traditionally intended to substantially release and/or deliver the active agent in the gastrointestinal tract beyond the mouth and/or buccal cavity.

The term “a therapeutically effective amount” of a compound, as used herein, refers to an amount that will elicit the biological or medical response of a subject, for example, ameliorate symptoms, alleviate conditions, slow or delay disease progression, etc. The term “a therapeutically effective amount” also refers to an amount of the compound that, when administered to a subject, is effective to at least partially alleviate and/or ameliorate a condition, a disorder, or a disease. The term “effective amount” means the amount of the subject compound that will engender a biological or medical response in a cell, tissue, organs, system, animal or human that is being sought by the researcher, medical doctor or other clinician.

The term “comprising” is used herein in its open ended and non-limiting sense unless otherwise noted. In a more limited embodiment “comprising” can be replaced by “consisting of”, which is no longer open-ended. In a most limited version it can include only feature steps, or values as listed in the respective embodiment.

### Abbreviations

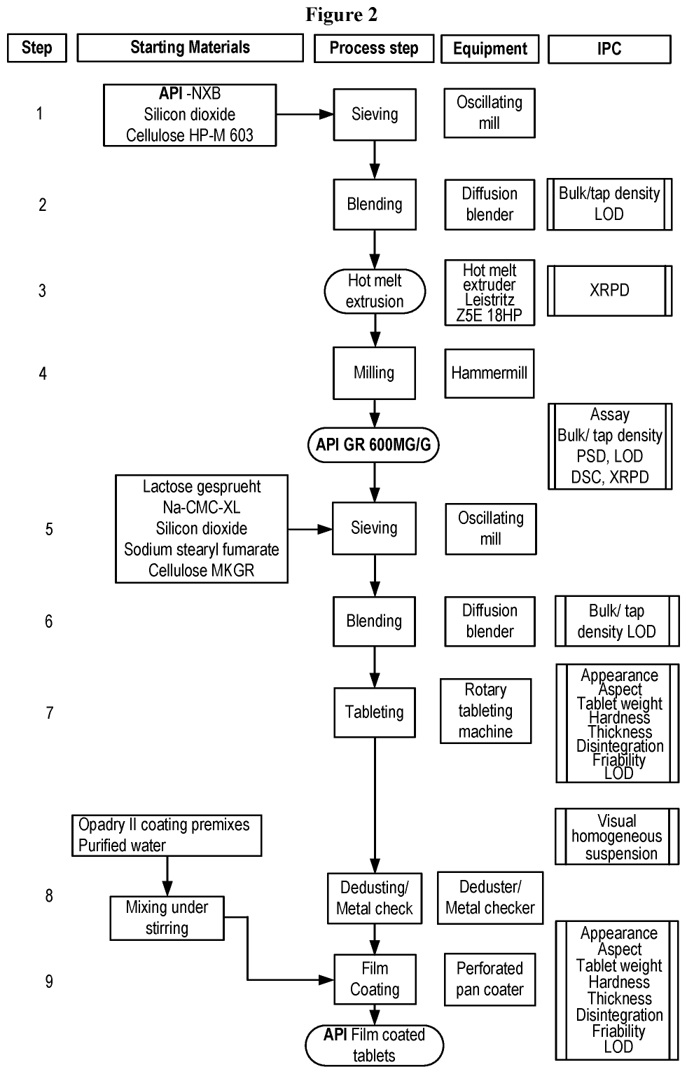

% w/w Percent weight by weight
° C. Degree Celsius
API Active pharmaceutical ingredient
API-NXB (or NXB) Compound A in the form of Monohydrate H_{A}
API-NXA (or NXA) Compound A in the form of anhydrate Form A
API-GR (from FIG. 2) Granules comprising Compound A
ASD Amorphous solid dispersion
AUC Area Under the Curve
AUCinf AUC curve up to infinite time
AUClast AUC up to the last measurable concentration
Cmax Maximum concentration
Cellulose HP-M 603 Hydroxypropyl methylcellulose
Cellulose MK GR Microcrystalline cellulose (MCC) granules
CV % Coefficient of variation (%)
CSF Clinical service form (formulation)
DR Dissolution rate
DSC Differential scanning calorimetry
FaSSIF Fasted state simulated intestinal fluid
FCT Film-coated tablet
FeSSIF Fed state simulated intestinal fluid
g/min Gram per minute
HME Hot-melt extrusion
HPLC High-Performance Liquid Chromatography
HR-XRPD High resolution-X-ray Powder Diffraction
INCI International Nomenclature of Cosmetic Ingredients
INN International nonproprietary name
IPC In-process controls
Kg/g/mg/ng/μg Kilogram/Gram/Milligram/Nanogram/Microgram
kN Kilo Newton
LCMS Liquid Chromatography-Mass Spectrometry
Lactose SD (or Lactose Lactose spray dried gesprueht in FIG. 2)
LOD Loss on drying
MEPC microemulsion pre-concentrate
MG/G milligram/gram
mL/L Milliliters/Liters
MRT Mean resistance time
Na-CMC-XL Sodium carboxymethyl cellulose
nm/μm Nanometer/Micrometer
PCS Photon correlation spectroscopy
Ph. Eur. European Pharmacopoeia
PK Pharmacokinetic
PSASD Polymer stabilized amorphous solid dispersion
PSD Particle size distribution
RH Relative humidity
Rpm Rotation per minutes
RRT Relative retention time
RT Room temperature
SD and RSD Standard deviation and relative standard deviation
SEM Scanning Electron Microscopy
SLS Sodium Lauryl sulfate
TFA Trifluoroacetic acid
TGA Thermogravimetric analysis
Tmax Time to peak maximum concentration (Cmax)
US Ultra sonication
USP United States Pharmacoepia
USP/NF United States Pharmacoepia/National Formulary
w/v weight by volume
w/w weight by weight
XRPD X-ray Powder Diffraction

### EXAMPLES

The following examples illustrate the invention and provide support for the disclosure of the present invention without limiting the scope of the invention.

### Example 1: Properties of Various Physical Forms of Compound A

Several physical forms of Compound A were analyzed: the free base, tartrate salt, and tosylate salt. A summary of the properties of these physical forms are provided in Table 1A.

TABLE 1A

| | Compound A | Compound A | Compound A |
|---|---|---|---|
| | (free base) | (tartrate salt) | (tosylate salt) |
| Stability in | Relatively stable at pH | Relatively stable at pH | Relatively stable at pH 2.9 to |
| aqueous | 2.9 to 6.8. | 2.9 to 6.8. | 6.8. |
| solutions | Significant degradation | Significant degradation | Significant degradation at |
| | at pH 1.2 and pH 10.0. | at pH 1.2 and pH 10.0. | pH 1.2 and pH 10.0. |
| Bulk stability | Physically unstable at | Physically unstable at | Physically unstable at 80° C./ |
| | 80° C./75% RH (1 | 80° C./75% RH (1 | 75% RH (1 week), no |
| | week), slight | week), significant | degradation. |
| | degradation. | degradation. | |
| Light stability | Slight degradation | Significant degradation | No degradation |
| (1.2 MLux h) | | | |
| Solubility at | SGF: 0.0021 (2.02) | SGF: 0.0535 (1.89) | SGF: 0.0100 (1.75) |
| 25° C. [mg/ml] | FaSSIF: 0.0014 (6.91) | FaSSIF: 0.0006 (3.54) | FaSSIF: 0.0011 (6.66) |
| (pH final) | FeSSIF: 0.1370 (6.08) | FeSSIF: 0.1092 (4.93) | FeSSIF: 0.1158 (5.58) |
| Hygroscopicity | Low | High | Low |
| Polymorphism | Yes | Potential issue | No issue encountered |

The tartrate salt form was found to be the least stable compound among the three and is hygroscopic. The free base was found to have comparable stability and hygroscopicity as the tosylate salt. But, the free base has at least two polymorphic forms while the tosylate salt has no observed polymorphism issue. The tosylate salt was not found to offer significant improvement in solubility in aqueous media and may have a potential toxicity risk in processing. The different physical forms of Compound A above show similar poor solubility

Compound A has very limited solubility in all pHs. Solubility of amorphous free base, crystalline hydrate and crystalline tosylate forms of Compound A are provided in Table 1B. Table 1B shows some pH dependent solubility profile but even at low pH, the solubility of Compound A is limited.

TABLE 1B

| Solubility [mg/ml] | Compound A | Compound A | Compound A |
|---|---|---|---|
| (pH final) | (free base) | (hydrate) | (tosylate) |
| pH 1 | 0.4 (1.07) | 0.17 (1.23) | 0.106 (1.23) |
| pH 2 | 0.14 (1.96) | — | — |
| pH 4.5 | 0.002 (4.56) | 0.001 (4.0) | 0.002 (3.85) |
| pH 7.0 | — | 0.0008 (7.0) | 0.002 (6.83) |
| pH 7.4 | 0.001 (7.33) | — | — |
| SGF pH 2.0 | 0.699 (1.86) | 0.025 (2.19) | 0.029 (2.02) |
| FaSSIF V2 | 0.035 (6.51) | 0.004 (6.44) | 0.016 (6.07) |
| FeSSIF V2 | 0.576 (5.82) | 0.047 (5.6) | 0.051 (5.41) |
| Water | 0.002 (7.33) | 0.001 (5.99) | 5.14 (2.71) |

The photostability of crystalline hydrate and crystalline tosylate forms of Compound A under photo, or light stress is provided in Table 1C. Table 1C shows that Compound A, as the crystalline hydrate and the crystalline tosylate salt, is stable as a bulk solid under light stress but is susceptible to degradation under light stress in solution form. Table 1D shows that Compound A, as the crystalline tosylate salt, is stable as a bulk solid under thermal stress at room temperature (RT), 50° C., and 80° C. for 5 days. Table 1E shows that Compound A, as the crystalline tosylate salt, is susceptible to degradation in solution/suspension form at low pH under heat.

TABLE 1C

| | | | | Degradation | Appearance/ |
|---|---|---|---|---|---|
| Compound A | | Temperature | Light dose | Products [%] | XRPD/ |
| (Form) | Medium | [° C.] | [Lux · h*10\hat{ }6] | (HPLC) | DSC |
| Hydrate | Bulk | RT | 1.2 | <1.0 | No change |
| Hydrate | Ethanol | RT | 1.2 | 18 | N/A |
| Tosylate | Bulk | RT | 1.2 | <1.0 | No change |
| Tosylate | Ethanol | RT | 1.2 | 97 | N/A |

TABLE 1D

| | | | Parent | |
|---|---|---|---|---|
| Compound A | Temperature | Exposure | remaining | Appearance/ |
| (Form) | [° C.] | Time | [%] (HPLC) | XRPD/DSC |
| Tosylate | RT | 5 days | >99 | No change |
| Tosylate | 50 | 5 days | >99 | No change |
| Tosylate | 80 | 5 days | >99 | No change |

TABLE 1E

| | | | Expo- | | |
|---|---|---|---|---|---|
| | | Temper- | sure | Parent | Appear./ |
| Compound A | | ature | Time | remaining | XRPD/ |
| (Form) | Medium | [° C.] | [d] | [%] (HPLC) | DSC |
| Tosylate | pH 1 | RT | 5 days | 97 | — |
| Tosylate | pH 1 | 50 | 5 days | 23 | — |
| Tosylate | pH 1 | 80 | 5 days | 0.2 | — |
| Tosylate | pH 4.5 | RT | 5 days | >99 | — |
| Tosylate | pH 4.5 | 50 | 5 days | >99 | — |
| Tosylate | pH 4.5 | 80 | 5 days | 98.6 | — |
| Tosylate | pH 7.4 | RT | 5 days | >99 | — |
| Tosylate | pH 7.4 | 50 | 5 days | 97 | — |
| Tosylate | pH 7.4 | 80 | 5 days | 74 | — |

Thus, it can be seen from the above that choosing which specific form of Compound A to process into an oral dosage form which is suitable for administration to a patient in need thereof is not a trivial exercise.

### Example 2: Pharmacokinetics of Compound a in Salt and Free Base Form Compositions

The pharmacokinetics of Compound A was investigated in dogs following a single oral dose of 100 mg/kg Compound A as tosylate salt in suspension with surfactant, free base in suspension with surfactant, and free base in microemulsion, as summarized in Table 2A and Table 2B. The dogs were fasted from normal feed overnight through approximately 4 hours post-dose for each phase. Each dog was dosed by oral gavage at 100 mg/kg (4 mL/kg) followed by 10 mL water flush. Compound A concentrations in plasma samples were quantified by liquid chromatography-tandem mass spectrometry.

TABLE 2A

| Study | | | Dose concentration | Dose |
|---|---|---|---|---|
| Arm | Form | Composition | & volume | (nominal) |
| 1 | Tosylate salt in | 1% HPC + 0.5% Pluronic F68 | 25 mg/mL, 4 mL/kg | 100 mg/kg |
| | suspension | | | |
| 2 | Free base in | 0.5% MC + 0.5% Tween 80 | 25 mg/mL, 4 mL/kg | 100 mg/kg |
| | suspension | | | |
| 3 | Free base in | MEPC4: Cremophor | 25 mg/mL, 4 mL/kg | 100 mg/kg |
| | microemulsion | RH40 45% + Corn oil | | |
| | (MEPC diluted) | glycerides 18% + PEG400 | | |
| | | 27% + Ethanol 10%. | | |

TABLE 2B

| | Formulation | | |
|---|---|---|---|
| | Arm 1 | Arm 2 | Arm 3 |
| PK Parameters | Mean ± SD | Mean ± SD | Mean ± SD |
| Tmax (h) | 1.7 ± 0.58 | 17 ± 13 | 1.7 ± 0.58 |
| Cmax (ng/mL) | 3290 ± 961 | 275 ± 148 | 7680 ± 839 |
| AUC0-24 h (h · | 48200 ± 20000 | 4370 ± 1900 | 93700 ± 28700 |
| ng/mL) | | | |
| AUC0-48 h (h · | 67000 ± 26900 | 8640 ± 4720 | 123000 ± 46300 |
| ng/mL) | | | |
| AUClast (h · | 72900 ± 31400 | 9410 ± 5240 | 133000 ± 53400 |
| ng/mL) | | | |
| T½ (h) | 14 ± 5.0 | 23 ± 14 | 14 ± 4.2 |
| AUCinf (h · ng/mL) | 76000 ± 34500 | 12200 ± 6530 | 138000 ± 56600 |
| AUCinf/dose ((h · | 760 ± 345 | 122 ± 65.3 | 1380 ± 566 |
| ng/mL)/(mg/kg)) | | | |

Overall, the free base in microemulsion yielded the highest AUC0-24 h (93700 h ng/mL) in dogs, followed by the tosylate salt (with AUC0-24 h=48200 h·ng/mL) in suspension and the free base (with AUC0-24 h=4370 h·ng/mL) in suspension. However, it was found that the composition in Study Arm 3, is a relatively unstable microemulsion.

### Example 3: Pharmacokinetics of Compound a in Various Formulations

The pharmacokinetics of Compound A were investigated in dogs following a single oral dose of 30 mg/kg Compound A as tosylate salt in suspension enriched with polymer (Phases A, B, C), as free base solid dispersion tablet (Phase D), and as free base microemulsion (Phase E), as summarized in Table 3A and Table 3B.

### Preparation of the Dosing Formulations

For Phases A-C, an appropriate amount of test drug substance, i.e., crystalline tosylate salt of Compound A, was weighed into a suitable container. Each formulation was prepared separately for each dog in a separate container by weighing 30 mg/kg dose (40.3 mg/kg tosylate salt) and adding vehicle of 0.2 M Na₂HPO₄ and 0.1 M citric acid aqueous solution at 3 mL/kg. In Phase A the vehicle was enriched with 1% (w/v) Eudragit® EPO, Phase B 1% (w/v) Hydroxypropyl cellulose (HPC), Phase C 1% (w/v) Kolliphor® RH40, respectively. The obtained suspensions were stored at ambient temperatures (18-30° C.) and administered within 15-30 minutes of formulation preparation. For Phase D, an amorphous solid dispersion tablet containing 300 mg of Compound A was prepared according to Example 10.

For Phase E, an active microemulsion pre-concentrate (MEPC) of Compound A was prepared at 100 mg/mL (Ingredients of passive MEPC: Ethanol, PEG400, Maisine CC, Kolliphor RH40). The formulation was prepared separately for each dog in a separate container by measuring 0.3 mL/kg Compound A MEPC to 0.7 mL/kg water to produce a microemulsion. The corresponding concentration was 30 mg/mL Compound A, 30 mg/kg dose. The formulations were stored at ambient temperatures (18-30° C.) and administered within 15-30 minutes of preparation.

The dogs were fasted from normal feed overnight through approximately 4 hours post-dose for each phase. For Phases A, B, C, the suspension formulations (3 mL/kg) were administered by oral gavage to six conscious dogs, followed by a gavage line flush with water at 2 mL/kg, total volume at 5 mL/kg. For Phase D, one tablet was orally administered per dog, followed by administration of pH 2.6 buffer by oral gavage at 3 mL/kg and a gavage line flush with water at 2 mL/kg, total volume at 5 mL/kg. During Phase E, the microemulsion (1 mL/kg) was administered by oral gavage to six conscious dogs, followed by an administration of pH 2.6 buffer by gavage at 3 mL/kg and a gavage line flush with water at 1 mL/kg, total volume at 5 mL/kg.

TABLE 3A

| | | | Free base | | | |
|---|---|---|---|---|---|---|
| | | | concentration in | Nominal | Formulation | Buffer |
| | | | formulation | dose | amount | amount |
| Phase | Formulation | Composition | (mg/mL) | (mg/kg) | (mL/kg) | (mL/kg) |
| A | Compound A | 1% (w/v) Eudragit | 10 | 30 | 3.0 | NA |
| | (tosylate) | EPO in phosphate | | | | |
| | Eudragit | citrate buffer, pH 2.6 | | | | |
| | suspension | | | | | |
| B | Compound A | 1% (w/v) HPC in | 10 | 30 | 3.0 | NA |
| | (tosylate) HPC | phosphate citrate | | | | |
| | suspension | buffer, pH 2.6 | | | | |
| C | Compound A | 1% (w/v) Kolliphor | 10 | 30 | 3.0 | NA |
| | (tosylate) | RH40 in phosphate | | | | |
| | Kolliphor | citrate buffer, pH 2.6 | | | | |
| | suspension | | | | | |
| D | Compound A | Tablet, in phosphate | N/A | 30 | N/A | 3.0 |
| | amorphous solid | citrate buffer, pH 2.6 | | | | |
| | dispersion | | | | | |
| | (ASD) tablet | | | | | |
| E | Compound A | Microemulsion in | 30 | 30 | 1.0 | 3.0 |
| | MEPC | phosphate citrate | | | | |
| | microemulsion | buffer, pH 2.6 | | | | |

TABLE 3B

| | Phase A | Phase B | Phase C | Phase D | Phase E |
|---|---|---|---|---|---|
| | Compound A Formulation | | | | |
| | Tosylate | | Tosylate | | |
| | suspension | | suspension | | |
| | with 1% | Tosylate | with 1% | | |
| | (w/v) | suspension | (w/v) | Free base | |
| | Eudragit | with 1% | Kolliphor | amorphous | |
| | EPO in | (w/v) HPC | RH40 in | solid dispersion | Free base |
| | phosphate | in phosphate | phosphate | (ASD) as 300 | microemulsion |
| | citrate buffer | citrate buffer | citrate buffer | mg tablet | 100 mg/mL |
| Mean PK Parameters | Mean ± SD | Mean ± SD | Mean ± SD | Mean ± SD | Mean ± SD |
| Actual Dose (mg/kg) | 30.2 ± 0.327 | 30.1 ± 0.373 | 30.2 ± 0.256 | 32.4 ± 6.35 | 30.3 ± 0.0623 |
| Cmax (ng/mL) | 144 ± 51.1 | 439 ± 144 | 530 ± 91.3 | 1120 ± 517 | 8680 ± 1690 |
| Cmax/D (ng · mL)/ | 4.77 ± 1.73 | 14.6 ± 4.88 | 17.6 ± 3.07 | 34.5 ± 13.3 | 286 ± 55.4 |
| (mg/kg) | | | | | |
| Tmax (h) | 2.0 (2.0-24) | 2.0 (1.0-4.0) | 1.5 (0.50-2.0) | 2.0 (2.0-4.0) | 1.0 (1.0-1.0) |
| Tlast (h) | 36 (24-96) | 72 (48-96) | 84 (48-96) | 96 (96-96) | 96 (96-96) |
| AUClast (h · ng/mL) | 2070 ± 1230 | 5040 ± 909 | 4230 ± 894 | 15600 ± 8710 | 68200 ± 3680 |
| AUClast/D (h · ng/mL)/ | 68.3 ± 39.8 | 167 ± 30.0 | 140 ± 29.4 | 476 ± 266 | 2250 ± 119 |
| (mg/kg) | | | | | |
| AUCinf (h · ng/mL) | 1680 ± 637 | 5200 ± 948 | 4740 ± 913 | 25200 (N = 2) | 68500 ± 3760 |
| AUCinf/D (h · ng/mL)/ | 55.6 ± 20.7 | 173 ± 30.8 | 157 ± 29.3 | 761 (N = 2) | 2260 ± 121 |
| (mg/kg) | | | | | |
| T½ (h) | 7.97 ± 4.25 | 9.86 ± 3.99 | 9.96 ± 3.79 | 16.1 (N = 2) | 15.9 ± 5.72 |

Following oral dose administration, serial blood samples were collected up to 96 h post dose. Following collection, each sample was centrifuged to generate plasma and all plasma samples were analyzed using a suitable LC-MS/MS assay, with a lower limit of quantification (LLOQ) of 1.0 ng/mL Compound A.

Comparing AUClast/dose, exposure after tablet administration of formulation D (amorphous solid dispersion formulation) was markedly greater (AUClast/D 476±266) than the exposure after oral gavage dosing of formulation A (Eudragit formulation) (AUClast/D 68.3±39.8), after oral gavage dosing of formulation C (AUClast/D 140±29.4), and after oral gavage dosing of formulation B (HPC formulation) (AUClast/D 167±30.0), while it was markedly less than the exposure after oral gavage dosing of formulation E (MEPC formulation) (AUClast/D 2250±119). However, the microemulsion was observed to be relatively unstable and furthermore, MEPC formulations may not be practical for use in therapy due to the need for ingestion of large amounts of lipid vehicles with each dose.

### Example 4: Pharmacokinetics of Compound A in ASD Formulations

Amorphous solid dispersion (ASD) formulations of Compound A were evaluated as follows. In a crossover study with dogs, using a nominal dose of 60 mg/kg, the pharmacokinetics of hot-melt extrusion (HME) solid dispersion and spray-dried (SD) solid dispersion as suspensions were evaluated against a micronized Compound A (API) suspension (as reference), as summarized in Table 4A and Table 4B. Hydroxypropyl methyl cellulose (HPMC/Hypromellose) was the stabilizing polymer used in the hot melt extrusion ASD. Copovidone (PVP VA64) and Eudragit® EPO were the stabilizing polymers used in the spray-dried ASD.

The dogs were fasted from normal feed overnight until after 4 hours post-dose. Each dog was conditioned with 2 mL/kg phosphate-citrate buffer pH 2.6 via oral gavage and the gavage line was flushed with 5 mL. The respective formulations were dosed immediately thereafter via oral gavage with 5 mL/kg of the each suspension and flushed with 5 mL of water to ensure the gavage tubing was cleared of the formulation.

TABLE 4A

| Study | | | | Dose concentration & |
|---|---|---|---|---|
| Arm | Form | Composition | Vehicle | volume |
| Phase 1 | Hot melt | 30% Compound A, | Water | 12 mg/mL (40 mg/mL |
| | extrusion | 67.5% HPMC E3, | | blend), 5 mL/kg |
| | amorphous | 2.5% Sorbitol | | |
| | solid | | | |
| | dispersion | | | |
| Phase 2 | Spray | 19.9% Compound A, | Water | 12 mg/mL (60.3 mg/mL |
| | dried solid | 39.8% PVP VA64 | | blend), 5 mL/kg |
| | dispersion | 39.8% EPO; 0.5% | | |
| | | silica | | |
| Phase 3 | Micronized | 99.5% Compound A, | 1% HPC, 0.5% | 12 mg/mL, 5 mL/kg |
| | API | 0.5% silica | Pluronic F68 | |
| | (reference) | | | |

TABLE 4B

| | Formulation | | |
|---|---|---|---|
| | Phase 1 | Phase 2 | Phase 3 |
| PK Parameters | Mean ± SD | Mean ± SD | Mean ± SD |
| Tmax (h) | 2.3 ± 1.5 | 3.3 ± 1.2 | 10 ± 12 |
| Cmax (ng/mL) | 2250 ± 923 | 2050 ± 159 | 572 ± 425 |
| AUC0-24 h | 37400 ± 20200 | 26700 ± 2320 | 7240 ± 5360 |
| (h · ng/mL) | | | |
| AUClast | 58900 ± 42900 | 35800 ± 4810 | 16100 ± 16300 |
| (h · ng/mL) | | | |
| Tlast (h) | 72 | 72 | 72 |
| Relative | 366 | 222 | 100 |
| bioavailability based | | | |
| on AUClast(%) | | | |

Relative to the micronized API formulation, the bioavailability of hot melt extrusion and spray-dried solid dispersion formulations were found to be higher by factor of 3.7 and 2.2, respectively. The hot melt extrusion and sray-dried amorphous solid dispersion formulations were comparable with no safety concerns related to excipients Improved pharmacokinetic attributes were observed with the HPMC based hot melt extrusion formulation. The spray-dried solid dispersion amorphous formulation was found to be poorly stable and could not easily be densified or compressed into tablet by roller compaction.

### Example 5: Clinical Service Formulation of Compound A

Drug-polymer mixtures of various compositions were prepared by hot melt extrusion (HME) in a microextruder and evaluated for amorphous stability and compatibility with excipients, for example as in Example 8.

Among the polymers evaluated, hypromellose-based (e.g., HPMC 2910) and copovidone-based ASDs with 30% drug loading were identified as the most suitable, in particular in terms of amorphous stability and compatibility with excipients. ASDs with these polymers were found to be amorphous by XRPD and remained physically and chemically stable upon short-term storage (1-2 weeks) and could be further developed into tablets of 50 mg strength. The HPMC-based tablet exhibited a faster rate of dissolution and greater recovery as compared to the copovidone-based tablet and sustained the supersaturation up to 2 hours.

A clinical service formulation (CSF-1), supplied as 50 mg (550 mg tablet) and 100 mg (1100 mg tablet) strength tablets having a dose-proportional composition containing 9.1% Compound A, 21% Hypromellose 2910, 55.6% microcrystalline cellulose, 10% crospovidone, 3.3% colloidal silica and 1% magnesium stearate, could be developed and used for further stability studies under International Council for Harmonization (ICH) guidelines and supportive shelf-life.

### Example 6: Animal Studies to Optimize Amorphous Solid Dispersion Compositions of Compound A

Three animal studies were conducted to elaborate an understanding of the in-vivo behavior of amorphous solid dispersion (ASD) compositions containing Compound A (API). In Dog Study 1, four different compositions having a drug load between 30-60% were administered to fasted dogs at doses of 30 mg/kg or 10 mg/kg. The dogs were pre-treated with phosphate-citrate buffer pH 2.6 and the compositions were dispersed in water and dosed. In Dog Study 2, three different compositions having a 60% drug load, were administered to fasted dogs at a dose of 30 mg/kg. The dogs were pre-treated with pentagastrin and the compositions were dispersed in water and dosed. In Dog Study 3, four different compositions were administered to fasted dogs. Formulations C1, C2, and C3 were dispersed in water and dosed at 10 mg/kg. Formulation C4 was administered as an intact tablet. Post administration the dogs were given a phosphate-citrate buffer pH 2.6 flush by oral gavage. A summary of the pharmacokinetic data for the dog studies are provided in Table 6.

TABLE 6

| | | Dose | | AUC last/dose | Cmax/dose |
|---|---|---|---|---|---|
| Study | Formulation | mg/kg | Vehicle | (h*ng/mL)/(mg/kg) | (ng/mL)/(mg/kg) |
| Dog | A1 | 30 (N = 3) | water | 2120 | 117 |
| study | 30% API, 69% HPMC, | | | | |
| 1 | 1% Aerosil 200 | | | | |
| | A2 | 30 (N = 3) | water | 1600 | 85.3 |
| | 40% API, 60% HPMC- | | | | |
| | AS-L | | | | |
| | A3 | 10 (N = 3) | water | 1930 | 156 |
| | 60% API, 40% HPMC | | | | |
| | A4 | 10 (N = 3) | water | 1870 | 133 |
| | 60% API, 40% HPMC- | | | | |
| | AS-L | | | | |
| Dog | B1 | 30 (N = 4) | water | 633 | 52.9 |
| study | 60% API, 37% HPMC- | | | (SD 349) | (SD 33.7) |
| 2 | AS-L, 3% SLS | | | | |
| | B2 | 30 (N = 4) | water | 570 | 39.5 |
| | 60% API, 30% HPMC- | | | (SD 385) | (SD 15.6) |
| | AS-L, 10% HPMC | | | | |
| | B3 | 30 (N = 4) | water | 645 | 54.7 |
| | 60% API, 10% HPMC- | | | (SD 422) | (SD 22.9) |
| | AS-L, 30% HPMC | | | | |
| Dog | C1 (A3) | 10 (N = 4) | water | 1110 | 105 |
| study | 60% API, 40% HPMC | | | (SD 497) | (SD 81.2) |
| 3 | C2 (B2) | 10 (N = 4) | water | 1090 | 112 |
| | 60% API, 30% HPMC- | | | (SD 860) | (SD 127) |
| | AS-L, 10% HPMC | | | | |
| | C3 (A1) | 10 (N = 4) | water | 998 | 93.2 |
| | 30% API, 69% HPMC, | | | (SD 162) | (SD 34.6) |
| | 1% Aerosil 200 (CSF-1 | | | | |
| | granules) | | | | |
| | C4 | 100 mg/dog | tablet | 935 | 100 |
| | 9.1% API, 21% HPMC, | (N = 4) | | (SD 496) | (SD 80.6) |
| | 55.6% MCC, 10% | | | | |
| | crospovidone, 3.3% | | | | |
| | colloidal silica, 1% | | | | |
| | magnesium stearate | | | | |
| | (CSF-1 tablet) | | | | |

In all studies, the inter-animal variability was moderate to high for all formulations. The AUClast and Cmax (mean) of API in plasma were comparable in all treatment groups within each study. However, marked differences were observed between the studies, e.g., the treatment (to normalize the stomach pH) of the dogs prior to dosing had significant impact on the exposure and plasma concentrations. In study 2, the dogs were pre-treated with 6 μg/kg pentagastrin and in study 1 and 3, 2 ml/kg phosphate citrate buffer (pH 2.6) were given before dose administration.

Based on these studies, the HPMC-based amorphous solid dispersion compositions with 60% drug loading were found to have optimum properties.

### Example 7: Impact of Drug Substance Characteristics on Drug Product Properties

Mixtures of a polymer such as hypromellose (HPMC) and Compound A in various physical forms (e.g., in the form of the anhydrate (referred to herein as “Compound A-NXA”) and in the form of the monohydrate H_{A} (referred to herein as Compound A-NXB)) were prepared as separate mixtures and processed into an amorphous solid drug dispersion using hot melt extrusion.

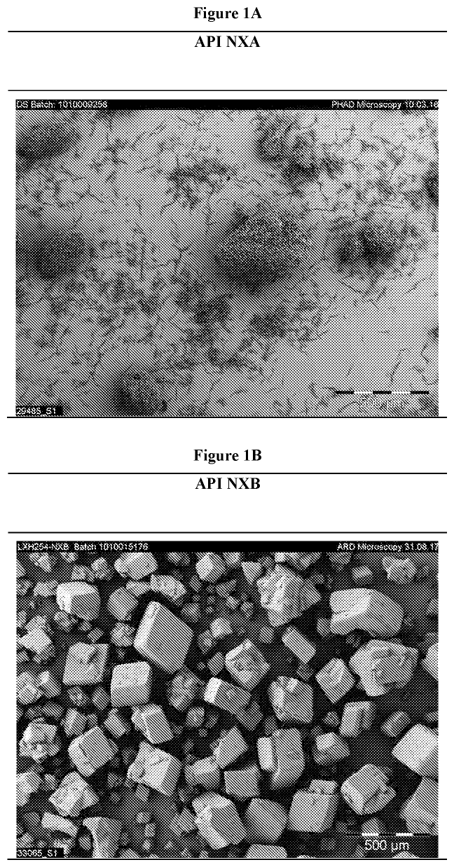

The bulk density of the pre-blend containing the anhydrate form of Compound A was 0.07-0.11 g/cm³ with flow function of 1.5-1.8. It was very cohesive and difficult to sustain uniform feeding into the extruder. The pre-blend containing the monohydrate form H_{A} of Compound A had a higher, more favorable, bulk density of 0.33 g/cm³ with a flow function of 2.2-2.3 and provided uniform feeding into the extruder. As depicted in FIGS. 1A and 1B, the anhydrate form of Compound A has very fine, needle-like crystal structure unlike Modification H_{A} of Compound A, which had a more cubic particle morphology.

Thus, it can be seen that amorphous solid dispersions prepared from Modification H_{A} of Compound A provide optimal flow of the pre-blend during processing, e.g., in the hot-melt extrusion process of the invention.

The invention thus provides for the use of a crystalline form of Compound A which is not in fine, needle-like shape, for use in a method of preparing an amorphous solid dispersion comprising Compound A.

### Example 8: Optimization of Tablet Formulations Containing Compound A

Optimization of the tablet formulations according to the invention can be carried out as follows.

In particular, it was found that certain tablets obtained using an amorphous solid dispersion prepared from Compound A in the anhydrate form and hypromellose suffered some physical defects. In some instances, hairline cracks were observed on the side wall of such a tablet after overnight storage. It was also difficult to allow for film-coating of such a tablet as there were some instances of lack of tablet content uniformity with the blend and fast tablet disintegration of 5-10 seconds. In addition, the tablets obtained using an amorphous solid dispersion prepared from Compound A in the anhydrate form and hypromellose could only accommodate a low drug loading, resulting in large tablets which are difficult to swallow. This leads to a large pill burden for the patients, particularly in cases where the recommended dose is high, and a resulting lack of patient compliance. For example, the total weight of a tablet containing only 9.1% of Compound A as drug load (100 mg of Compound A) was 1100 mg, The tablet size of a tablet obtained from an ASD prepared from the anhydrate form of Compound A was large (20×10.6 mm).

Surprisingly, the drug loading in the amorphous solid dispersions could be significantly increased, by using Monohydrate H_{A} of Compound A to prepare the amorphous solid dispersion. Thus, the drug load in the amorphous solid dispersion comprising Compound A could be doubled (from about 30% to 60%), the tablet size could be made significantly smaller (up to 70% reduction) for a 200 mg tablet (17×6.7 mm) as compared to the 100 mg tablet obtained from an amorphous solid dispersion using Compound A in the anhydrate form (20×10.6 mm). The tablet made from an amorphous solid dispersion prepared from Compound A in the Monohydrate H_{A} form was also physically robust enough for a film coating to be applied.

Amorphous solid dispersions prepared with Monohydrate H_{A} form of Compound A and various polymers (HPMC 2910, HPMC-AS-L, HPMC-AS-H, Eudragit® L100-55), melt extruded at three different drug loads (40%, 60%, and 80%) and milled to powders, were investigated for stability and dissolution rate testing.

The two most promising PSASD powders (60% API/40% HPMC 2910 and 60% API/30% HPMC-AS-L/10% HPMC 2910) were further developed into tablets for evaluation on adequate compactibility/processability, fast to moderately fast disintegration time, compatibility with excipients, and suitability for film coating. A unit dose range varying from 50 to 300 mg was investigated for both variants. The HPMC variant was selected based on its improved chemical stability and compatibility.

Film-coated tablets (e.g., 50 and 200 mg strength tablets) could be developed and coated with Opadry II.

Several weaknesses, mostly related to processability, compressibility and in vitro performance (disintegration time/dissolution rate) identified with the CSF-2 tablet formulation were addressed with the FMI tablet formulation. Silicon dioxide was added to the extrudate together with HPMC and allowed for better milling attributes of extrudate, better compressibility profiles and improvement of disintegration time. In addition, microcrystalline cellulose was added to the external phase of the final blend. These adaptations provided a more suitable particle size distribution of the extrudate and an overall improved tablet compression. In addition, the sodium bicarbonate and crospovidone in CSF-2 tablet was replaced with croscarmellose sodium to facilitate tablet disintegration.

Compositions of 100 mg CSF1, 200 mg CSF2, and 200 mg FMI tablets are provided in Table 8. The drug product manufacturing process involves unit operations of pre-blending drug and polymer, hot-melt extrusion, pelletization and milling to obtain the powdered amorphous solid dispersion (ASD). This is followed by final blending with excipients and lubricants, tablet compression and film-coating. No special packaging or device was needed for tablet development as a solid dosage form.

TABLE 8

| | Amount per unit [mg] | | | |
|---|---|---|---|---|
| | 100 mg | 200 mg | 200 mg | |
| Component | CSF1 | CSF2 | FMI^{b} | Function |
| API-NXA or NXB^{a} | 100.00 | 200.00 | 200.00 | Active ingredient |
| HPMC | 230.00 | 133.30 | 123.33 | Dispersing agent |
| Silicon dioxide | 3.34 | — | 10.00 | Glidant |
| Granulate weight | 333.34 | 333.30 | 333.33 | |
| Microcrystalline | 612.66 | — | 133.00 | Diluent |
| cellulose | | | | |
| Lactose | — | 213.87 | 88.67 | Diluent |
| Crospovidone | 110.00 | 51.20 | — | Disintegrant |
| Croscarmellose sodium | — | — | 30.00 | Disintegrant |
| Magnesium stearate | 11.00 | — | — | Lubricant |
| Sodium bicarbonate | — | 25.60 | — | Disintegrant |
| Sodium stearyl fumarate | — | 9.60 | 9.00 | Lubricant |
| Silicon dioxide | 33.00 | 6.40 | 6.00 | Glidant |
| Tablet/core fill weight | 1100.00 | 640.00 | 600.00 | |
| Opadry II Yellow | — | 13.57 | 6.44 | Film Coating |
| Opadry II White | — | 4.14 | 9.95 | Film Coating |
| Opadry II Red | — | 4.14 | 1.47 | Film Coating |
| Opadry II Black | — | 1.15 | 0.14 | Film Coating |
| Film coated tablet (FCT) | — | 663.00 | 618.00 | |
| weight | | | | |

^{a}NXA (anhydrous Form A) form used in CSF1, whereas NXB (monohydrate HA) used in CSF2 and FMI.
^{b}100 mg FMI is dose proportional to 200 mg composition

### Example 9: FCT Compositions of Compound A

FIG. 2 illustrates a representative process flow diagram for manufacturing 600 mg/g granules of Compound A (API) and the addition of extra-granular components for manufacturing a film coated tablet (FCT) of Compound A (API).

In FIG. 2, HPM 603 refers to “HPMC603” which is also known as HPMC 2910.

Granules comprising 60 weight % of Compound A, where the weight % refers to the weight of A compared to the total weight of the granules can be prepared according to Table 9A.

The hot-melt extrusion was performed with a batch size of 25 kg pre-blend using the Leistritz 18 mm twin-screw extruder. The conditions for hot-melt extrusion are provided in Table 9B.

TABLE 9A

| | |
|---|---|
| FCT 50 MG | FCT 300 MG |

| Ingredient | % | mg | % | mg |
|---|---|---|---|---|
| API-NXB | 33.33 | 50.00 | 33.33 | 300.00 |
| HPMC | 20.56 | 30.83 | 20.56 | 185.00 |
| Silicon dioxide | 1.67 | 2.50 | 1.67 | 15.00 |
| API GR | 55.56 | 83.33 | 55.56 | 500.00 |
| 600 MG/G | | | | |
| Microcrystalline | 22.17 | 33.25 | 22.17 | 199.50 |
| cellulose | | | | |
| Lactose | 14.78 | 22.17 | 14.78 | 133.00 |
| Croscarmellose | 5.00 | 7.50 | 5.00 | 45.00 |
| sodium | | | | |
| Silicon dioxide | 1.00 | 1.50 | 1.00 | 9.00 |
| Sodium stearyl | 1.50 | 2.25 | 1.50 | 13.50 |
| fumarate | | | | |
| Total TABLET | 100.01 | 150.00 | 100.01 | 900.00 |
| Opadry II white | | 3.87 | | 12.72 |
| Opadry II yellow | | 2.51 | | 8.23 |
| Opadry II red | | 0.57 | | 1.88 |
| Opadry II black | | 0.05 | | 0.17 |
| Total FCT | | 157.00 | | 923.00 |

TABLE 9B

| Process conditions | API GR 600 MG/G |
|---|---|
| Zone temperatures | |
| Z1 to 9 mm Die | 25° C. to 170° C. |
| Screw speed [rpm] | 150-350 |
| Torque [%] | 17-35 |
| Die pressure [bar] | 8.45-12.47 |
| Feed rate [kg/h] | 1.0-5.0 |
| Specific energy input (kWh/kg) | 0.19-0.32 |
| Chill roll | |
| Calendar speed (Hz) | 65 |
| Kibber speed (Hz) | 60 |
| Water temp. (° C.) | 13.0-13.6 |
| Roller gap (mm) | 0.21 |

After extrusion, the extrudates were milled with a Frewitt hammer mill (hammer forward). The milled extrudates are tested for granule assay, bulk/tap density, particle size distribution (PSD), loss on drying (LOD), differential scanning calorimetry (DSC), and x-ray powder diffraction (XRPD) in accordance to acceptance criteria.

In total 10 kg of milled extrudates were used for the preparation of the final blend. For the compression, a total amount of 17.69 kg was available and split for the compression of the 50 mg (6 kg final blend=40′000 units) and 300 mg (11.5 kg final blend=12′777 units) dose strength. The compression into tablets was performed on a rotary press (Fette 1200i) equipped with 8 punches.

### Example 10: Manufacturing Process Intermediate—Granules of Compound A

FIG. 2 illustrates a representative process flow diagram for manufacturing 600 mg/g granules of Compound A (API) and the addition of extra-granular components for manufacturing a film coated tablet (FCT) of Compound A (API).

The batch formula of Table 10A is representative for 1 kg of granules of Compound A (API GR). The process conditions for hot-melt extrusion are provided in Table 10B. The batch size of the granules (used as an intermediate) will depend upon clinical requirements and/or available starting materials. The weight of individual components corresponds proportionally to the stated composition.

TABLE 10A

| | API GR 600 MG/G | | | |
|---|---|---|---|---|
| | | | | Dispensed |
| Ingredient | % | mg | kg/batch | kg/batch* |
| API-NXB ** | 60.00 | 600.00 | 5.771 | 6.030 |
| HPMC | 37.00 | 370.0 | 3.559 | 3.559 |
| Silicon dioxide | 3.00 | 30.00 | 0.289 | 0.289 |
| API GR 600 MG/G | 100.00 | 1000.00 | 9.619 | 9.878 |

*Adjusted amounts based on drug content in Example 9 (Drug content = 95.7%)
The API-NXB drug substance is expressed as dry free base.

The 600 mg/g API intermediate was prepared following a procedure as described in the flowchart of FIG. 2. In step 1, the components are sieved in the following order into a suitable container: API-NXB, silicon dioxide, HPMC. In step 2, the mixture of step 1 is blended. In step 3, hot-melt extrusion of the mixture is performed. In step 4, the melt extrudates from step 3 are milled to form granules.

TABLE 10B

| Process conditions | API GR 600 MG/G |
|---|---|
| Zone temperatures | |
| Z1 to 5 mm Die | 25° C. to 160° C. |
| Screw speed [rpm] | 150-200 |
| Torque [%] | 33-39 |
| Die pressure [bar] | 15.1-19.6 |
| Feed rate [kg/h] | 4.0-5.0 |
| Specific energy input (kWh/kg) | 0.21-0.32 |
| Chill roll | |
| Calendar speed (Hz) | 65 |
| Kibber speed (Hz) | 60 |
| Water temp. (° C.) | 13 |
| Roller gap (mm) | 0.5 |

The extrusion could be started with 3 kg/h feed rate and 150 rpm screw speed and afterwards continuously increased to 5 kg/h feed rate and 200 rpm screw speed. Moreover, the water temperature at the chill roll and the roll gap was increased from 15° C. and 0.21 mm to 17-18° C. and 0.5 mm. With water temperatures ≤15° C. the extrudate film started to stick due to condensation on the chill roll.

TABLE 10C

| Process conditions | API GR 600 MG/G |
|---|---|
| Zone temperatures | |
| Z1 to 5 mm Die | 25° C. to 160° C. |
| Screw speed [rpm] | 150-200 |
| Torque [%] | 31-39 |
| Die pressure [bar] | 14.4-19.1 |
| Feed rate [kg/h] | 3.0-5.0 |
| Specific energy input (kWh/kg) | 0.27-0.33 |
| Chill roll | |
| Calendar speed (Hz) | 62 |
| Kibber speed (Hz) | 60 |
| Water temp. (° C.) | 16.7-17.8 |
| Roller gap (mm) | 0.5 |

### Example 11: FMI FCT Compositions of Compound A

In accordance the manufacturing process intermediate of Example 10, four extrusion batches with a batch size of 17 kg were manufactured and further processed into three drug product batches (1×100 mg and 2×200 mg) with a batch size of 80,000 units. A representative process flow for manufacturing 100 mg and 200 mg film-coated tablet (FCT) compositions of Compound A in Table 11A is illustrated in FIG. 2.

TABLE 11A

| | |
|---|---|
| FCT 100 MG | FCT 200 MG |

| Ingredient | % | mg | % | mg |
|---|---|---|---|---|
| API-NXB* | 33.33 | 100.000 | 33.33 | 200.000 |
| HPMC | 20.56 | 61.666 | 20.56 | 123.333 |
| Silicon dioxide | 1.67 | 5.000 | 1.67 | 10.000 |
| API GR | 55.56 | 166.666 | 55.56 | 333.333 |
| 600 MG/G | | | | |
| Cellulose MK | 22.17 | 66.500 | 22.17 | 133.000 |
| GR** | | | | |
| Lactose SD** | 14.78 | 44.334 | 14.78 | 88.667 |
| Croscarmellose | 5.00 | 15.000 | 5.00 | 30.00 |
| sodium** | | | | |
| Silicon dioxide** | 1.00 | 3.000 | 1.00 | 6.000 |
| Sodium stearyl | 1.50 | 4.500 | 1.50 | 9.000 |
| fumarate | | | | |
| Total TABLET | 100.01 | 300.000 | 100.01 | 600.000 |
| Opadry II | | 10.747 | | 14.612 |
| white*** | | | | |
| Opadry II | | — | | 2.819 |
| yellow*** | | | | |
| Opadry II red*** | | 0.0935 | | 0.522 |
| Opadry II | | 0.1595 | | 0.047 |
| black*** | | | | |
| Total FCT | | 311.000 | | 618.000 |

| | | |
|---|---|---|
| Coating color | Light greyish violet | Pale brown |

The API-NXB drug substance is expressed as dry free base.
***The film coating suspension is prepared with 20% solids. The coating suspension is prepared with overage to account for spray losses and losses in the spray system. For the film coating the batch may be coated in sub-batches based on the coater capacity and available coating pan.

1. An amorphous solid dispersion comprising an N-(3-(2-(2-hydroxyethoxy)-6-morpholinopyridin-4-yl)-4-methylphenyl)-2-(trifluoromethyl)isonicotinamide, or a pharmaceutically acceptable salt thereof, and one or more stabilizing polymers, wherein the weight ratio of Compound A, or a pharmaceutically acceptable salt thereof, to one or more stabilizing polymers is between about 5:95 to about 90:10, about 40:60, about 80:20; preferably about 60:40.

2. The amorphous solid dispersion according to claim 1, wherein the amorphous solid dispersion is made by spray drying, co-grinding, hot-melt extrusion, freeze drying, rotary evaporation, solvent evaporation, co-precipitation, lyophilization, or any suitable solvent removal process, preferably, hot-melt extrusion.

3. The amorphous solid dispersion according to claim 2, wherein the amorphous solid dispersion is prepared from N-(3-(2-(2-hydroxyethoxy)-6-morpholinopyridin-4-yl)-4-methylphenyl)-2-(trifluoromethyl)isonicotinamide in an amorphous form, in a crystalline form, or in a mixture thereof.

4. The amorphous solid dispersion according to claim 3, wherein N-(3-(2-(2-hydroxyethoxy)-6-morpholinopyridin-4-yl)-4-methylphenyl)-2-(trifluoromethyl)isonicotinamide is in the crystalline Monohydrate Form H_{A} characterized by having an X-ray powder diffraction pattern with at least one, two, three, four or five peaks having an angle of refraction 2 theta (θ) values selected from 7.3, 10.7, 16.3, 16.7, 17.4, 23.0, 24.3, 25.3, 28.3, 32.0 when measured using CuKα radiation, wherein said values are plus or minus 0.2° 2θ.

5. The amorphous solid dispersion according to any one of claims 1 to 4, wherein the one or more stabilizing polymers is selected from the group consisting of polyvinyl pyrrolidone (povidone or PVP), polyvinylpolypyrrolidone (crospovidone or PVP-XL), hydroxypropyl cellulose (HPC), low-substituted hydroxypropyl cellulose (L-HPC), hypromellose (HPMC), hypromellose acetate succinate (HPMC-AS), hypromellose phthalate (HPMC-P), carboxymethyl cellulose, croscarmellose sodium (NaCMC), methyl cellulose, hydroxyethyl cellulose, carboxyethyl cellulose, carboxymethyl cellulose, carboxymethylhydroxyethyl cellulose, polyethylene glycol (PEG), polyvinylalcohol, polyvinylpyrrolidone-vinyl acetate copolymer (copovidone or PVP/VA), polyvinyl alcohol-polyethylene glycol co-polymer, polyvinyl caprolactam-polyvinyl acetate-polyethylene glycol graft copolymer, polyacrylates, polymethacrylates, or a mixture thereof.

6. The amorphous solid dispersion according to claim 5, wherein the stabilizing polymer is HPMC, preferably HPMC 2910.

7. The amorphous solid dispersion according to any one of claims 1 to 6, further comprising a glidant selected from the group consisting of silicon dioxide, stearic acid, magnesium stearate, calcium stearate, talc, hydrogenated castor oil, sucrose esters of fatty acid, microcrystalline wax, yellow beeswax, white beeswax, and the like and mixtures thereof, preferably silicon dioxide, more preferably colloidal silicon dioxide.

8. The amorphous solid dispersion according to any one of claims 1 to 7, further comprising a solubilizer selected from the group consisting of polyoxyethylene alkylaryl ethers, polyethylene glycol fatty acid esters, D-α-tocopheryl polyethylene glycol succinate, polyoxyethylene sorbitan fatty acid ester, alkyl sulfates or sulfonates, lecithin, polyethoxylated castor oils and the like and mixtures thereof.

9. The amorphous solid dispersion according to any one of claims 1 to 8, wherein N-(3-(2-(2-hydroxyethoxy)-6-morpholinopyridin-4-yl)-4-methylphenyl)-2-(trifluoromethyl)isonicotinamide, or a pharmaceutically acceptable salt thereof, is from about 1% to about 90% (w/w), from about 10% (w/w) to about 85% (w/w), preferably from about 15% (w/w) to about 80% (w/w), from about 20% (w/w) to about 75% (w/w), or from about 30% (w/w) to about 60% (w/w) of the dispersion.

10. The pharmaceutical composition comprising an amorphous solid dispersion according to any one of claims 1 to 9 and optionally one or more pharmaceutically acceptable excipient(s) selected from solubilzers, diluents, binders, disintegrants, fillers, lubricants, glidants, surfactants, stabilizing agents, antioxidants, alkaline stabilizers, colors, flavors, preservatives, and combinations thereof.

11. The pharmaceutical composition according to claim 10 wherein the pharmaceutical composition comprises from about 10 mg to about 300 mg of N-(3-(2-(2-hydroxyethoxy)-6-morpholinopyridin-4-yl)-4-methylphenyl)-2-(trifluoromethyl)isonicotinamide, or a pharmaceutically acceptable salt thereof, preferably 50 mg, 100 mg, 200 mg, or 300 mg of N-(3-(2-(2-hydroxyethoxy)-6-morpholinopyridin-4-yl)-4-methylphenyl)-2-(trifluoromethyl)isonicotinamide, or a pharmaceutically acceptable salt thereof.

12. The pharmaceutical composition according to any one of claims 10 to 11, wherein the pharmaceutical composition is in the form of a tablet, a capsule, a caplet, beads, granules, oral suspension, oral solution, or microemulsion.

13. A pharmaceutical composition according to any one of claims 10 to 12, wherein the pharmaceutical composition is in the form of a tablet or a capsule comprising: (a) an amorphous solid dispersion of Compound A in the form of granules, (b) at least one intra-granular excipient, (c) at least one extra-granular excipient, and (d) optionally, a coating.

14. The pharmaceutical composition according to claim 13, wherein the extra-granular excipients are selected from solubilizers, diluents, binders, disintegrants, fillers, lubricants, glidants, surfactants, stabilizing agents, antioxidants, alkaline stabilizers, colors, flavors, preservatives, and combinations thereof.

15. The pharmaceutical composition according to claim 14, wherein the extra-granular excipients comprise diluents selected from the group consisting of microcrystalline cellulose, calcium carbonate, calcium phosphate-dibasic, calcium phosphate-tribasic, calcium sulfate, cellulose powdered, dextrates, dextrins, dextrose excipients, fructose, kaolin, lactitol, lactose, mannitol, sorbitol, starch, starch pregelatinized, sucrose, sugar compressible, sugar confectioners and combinations thereof, preferably lactose, microcrystalline cellulose, or lactose and microcrystalline cellulose.

16. The pharmaceutical composition according to claim 14, wherein the extra-granular excipients comprise disintegrants selected from the group consisting of croscarmellose sodium, low-substituted hydroxypropyl cellulose (L-HPC), polyvinylpolypyrrolidone (crospovidone), sodium bicarbonate, sodium starch glycollate, carboxymethyl cellulose, calcium carboxymethyl cellulose, sodium carboxymethyl cellulose, starch, crystalline cellulose, hydroxypropyl starch, pregelatinized starch, and mixtures thereof, preferably sodium bicarbonate and crospovidone, more preferably croscarmellose sodium.

17. A method for preparing an amorphous solid dispersion according to any one of claims 1 to 9 or a pharmaceutical composition according to any one of claims 10 to 16, which comprises preparing a mixture of N-(3-(2-(2-hydroxyethoxy)-6-morpholinopyridin-4-yl)-4-methylphenyl)-2-(trifluoromethyl)isonicotinamide, or a pharmaceutically acceptable salt thereof, one or more stabilizing polymers, and optionally one or more pharmaceutically acceptable excipients; heating the mixture to form a molten mass; extruding the molten mass; cooling the molten mass to form an amorphous solid dispersion; and optionally the granulating the amorphous solid dispersion and/or compacting granules of the amorphous solid dispersion for further processing with optionally one or more pharmaceutically acceptable excipients to form a composition suitable for use in dosage forms, preferably a tablets or a capsule.

18. The pharmaceutical composition according to any one of claims 10 to 16 for use as a medicament.

19. The pharmaceutical composition according to any one of claims 10 to 16 for use in the treatment of cancer.

20. The pharmaceutical composition according any one of claims 10 to 16 for use in the treatment of cancer, in particular in the treatment of cancers harboring MAPK pathway alterations such as KRAS-mutant NSCLC (non-small cell lung cancer), KRAS-mutant pancreatic cancer (e.g., KRAS-mutant pancreatic ductal adenocarcinoma (PDAC)), KRAS-mutant CRC (colorectal cancer), and NRAS-mutant melanoma.

21. A method of treating cancer comprising administering to a subject in need thereof a therapeutically effective amount of the pharmaceutical composition according to any one of claims 10 to 16.

22. The method of claim 21, wherein the cancer is harboring MAPK pathway alterations such as KRAS-mutant NSCLC (non-small cell lung cancer), KRAS-mutant pancreatic cancer (e.g., KRAS-mutant pancreatic ductal adenocarcinoma (PDAC)), KRAS-mutant CRC (colorectal cancer), and NRAS-mutant melanoma.
